(19) United States
(12) Patent Application Publication
(10) Pub. No.: US 2024/0158755 A1
(43) Pub. Date: May 16, 2024

(54) 3D HUMAN DISTAL LUNG ORGANOIDS CONTAINING EPITHELIAL, IMMUNE AND MESENCHYMAL COMPONENTS

(71) Applicant: The Board of Trustees of the Leland Stanford Junior University, Stanford, CA (US)

(72) Inventors: Calvin J. Kuo, Palo Alto, CA (US); Shannon Choi, Redwood City, CA (US)

(21) Appl. No.: 18/289,881

(22) PCT Filed: May 18, 2022
(86) PCT No.: PCT/US2022/029869

§ 371 Date: Nov. 7, 2023

Related U.S. Application Data
(60) Provisional application No. 63/190,147, filed on May 18, 2021.

(51) Int. Cl. C12N 5/071; G01N 33/50

(52) U.S. Cl. CPC C12N 5/0688; G01N 33/5082; C12N 2501/11; C12N 2502/11; C12N 2502/1352; C12N 2513/00

(57) ABSTRACT

Compositions and methods are provided for the culture of mammalian distal lung organoids that robustly preserve and propagate distal lung alveoli and bronchioles en bloc with infiltrating endogenous immune cells and mesenchyme as a cohesive unit without artificial reconstitution, allowing analysis of the dynamics of tissue-level inflammation upon respiratory infection.

### CROSS REFERENCE TO RELATED APPLICATION

The present application claims the benefit of PCT Application No. PCT/US2022/029869, filed May 18, 2022, and claims priority to U.S. Provisional Patent Application No. 63/190,147, filed May 18, 2021, the entire disclosure of which is hereby incorporated by reference in its entirety.

### STATEMENT REGARDING FEDERALLY SPONSORED RESEARCH

This invention was made with Government support under contract AI116484 awarded by the National Institutes of Health. The Government has certain rights in the invention.

### INCORPORATION BY REFERENCE OF SEQUENCE LISTING

A Sequence Listing is provided herewith as a Sequence Listing text, “STAN-1852_SEQUENCE_LISTING_ST25” created on Nov. 7, 2023 and having a size of 3,864 bytes. The contents of the Sequence Listing text are incorporated herein by reference in their entirety.

### BACKGROUND

Respiratory infections are a frequent causative agents of disease in humans, with significant impact on morbidity and mortality worldwide. Common respiratory agents from several virus families are well adapted to efficient person-to-person transmission and circulate in a global scale, and community-based studies conducted over the past five decades or so confirm that these viruses are the predominant etiological agents of acute respiratory infections. The respiratory viruses that most commonly circulate in all continents as endemic or epidemic agents are influenza virus, respiratory syncytial virus, parainfluenza viruses, metapneumovirus, rhinovirus, coronaviruses, adenoviruses, and bocaviruses.

The distal lung is a complex unit that contains not only respiratory epithelium (alveoli, terminal bronchioles), but also diverse mesenchymal stromal components (c.f. fibroblasts, endothelium, others) and immune populations (T and B cells, macrophages, others). The interaction between lung epithelium, mesenchymal stroma and immune cells underlies essentially all lung pathologies, inclusive of infection, cancer and autoimmunity, for instance with cytokine storm and SARS-CoV-2 infection or anti-tumor immunity in cancer. Historically, however, cultures of distal human lung have only included epithelium and not these other cell types and therefore are incapable of modeling the interactions between epithelium, mesenchyme and immune cells. Despite the unquestioned role of inflammation during infectious disease pathogenesis, human in vitro pathogen models have not incorporated a full diversity of tissue resident immune populations.

In the current COVID-19 pandemic, mortality is strongly associated with pathologic pulmonary inflammation and resultant respiratory failure. Many studies have investigated systemic immunity in COVID-19 patients by analyzing circulating cytokines, antibody production and T cell responses. However, definition of SARS-CoV-2-induced network immune interactions within the lung parenchyma, particularly during early stage infection as opposed to end stage disease, remains comparatively elusive. Rodent COVID-19 models are limited by interspecies host-pathogen incompatibility where SARS-CoV-2 infection requires either spike protein mutation to facilitate mouse ACE2 binding or transgenic expression of human ACE2. Additionally, many in vitro systems of SARS-CoV-2 pathogenesis have utilized immortalized cell lines that do not accurately recapitulate the cellular diversity of primary tissue. This knowledge gap has afforded relatively few treatment options for COVID-19, in part due to lack of human in vitro systems capable of modeling immune cell interaction with lung epithelium upon SARS-CoV-2 infection.

Organoids, or 3-dimensional tissue culture systems, have recently emerged as promising ex vivo models. Derivable from wild-type primary human tissue, organoids can retain the diverse cellular makeup of in vivo tissue epithelium. Development of organoids for modeling infectious disease processes is of great interest. However, organoids that retain endogenous immune populations in continuity with the more typical epithelial cells have been elusive, and no such immune:epithelial organoid systems have existed for lung.

### SUMMARY

Compositions and methods are provided for the culture of mammalian distal lung organoids that robustly preserve and propagate distal lung alveoli and bronchioles en bloc with infiltrating endogenous immune cells and mesenchyme as a cohesive unit without artificial reconstitution, allowing analysis of the dynamics of tissue-level inflammation upon respiratory infection. The lung organoids may be cultured from human lung tissue. The organoids recapitulate the cellular architecture and ultrastructure of the lung sample from which they were derived, and include immune cells such as lung infiltrating lymphocytes, parenchymal and stromal elements. The organoids optionally have everted polarity, in which differentiated club and ciliated cells are relocated from the organoid lumen to the exterior surface, thus displaying the ACE2 receptor on the outwardly-facing apical aspect, which can be infected with pathogens that utilize the ACE2 receptor, including without limitation SARS-CoV-2.

The organoid cultures are used for modeling lung pathologies, including, for example, infection, autoimmunity, cancer, etc.; and for screening therapeutics to treat such pathologies; for precision medicine; and stem cell-based therapies. Screening assays useful to determine complex responses to therapies, including but not limited to antiviral therapies, anti-inflammatory therapies, immune therapies, and the like are provided.

In some embodiments methods are provided for modeling respiratory infection-induced signaling between epithelium and immune cells, within immune cells, and their reciprocal feedback to epithelium. In some embodiments the infection is a viral infection, e.g. coronavirus including SARS-CoV1, SARS-CoV2, etc.; influenza virus; respiratory syncytial virus (RSV); parainfluenza virus; etc. In some embodiments the infection is a bacterial infection, e.g. M. tuberculosis complex, Legionella, etc. In such embodiments, an organoid of the disclosure is infected with a respiratory pathogen, and the effect on epithelial an immune cells is monitored, including monitoring over a time course, monitoring in the absence or presence of a therapeutic agent; and the like. Methods of analysis include, without limitation, single cell RNA sequencing of epithelial and immune components, microscopy, including fluorescence microscopy and staining, H&E and 3D confocal imaging with staining for differentiation and polarity markers; quantitative RT-PCR, e.g. to measure the accumulation of viral genomes or mRNA after infection; spatial imaging analysis of stained markers in FFPE sections at single cell resolution, and the like. The viability of lung epithelial and immune cells can be monitored throughout the infection process, e.g. by annexin V staining, etc.

In some embodiments, drug screening is performed using SARS-CoV-2 or other respiratory infectious pathogens, e.g. bacteria, viruses, and the like. In some aspects, a method is provided for in vitro screening for agents for their effect on cells of different cell types, e.g. epithelial, immune, stromal present in an organoid, including processes of viral infection initiation and treatment. Organoids cultured by the methods described herein are exposed to candidate agents, optionally before or after exposure to a respiratory pathogen. Agents of interest include pharmaceutical agents, e.g. known therapeutic agents; small molecules, antibodies, peptides, etc., and genetic agents, e.g. antisense, RNAi, expressible coding sequences, and the like, e.g. expressible coding sequences for candidate secreted growth factors, cytokines, receptors or inhibitors thereof, or other proteins of interest, and the like. In some embodiments the effect of candidate therapeutic agents on viral infection-related immune responses or their downstream effects is determined, for example where agents may include, without limitation, chemotherapy, monoclonal antibodies or other protein-based agents, radiation/radiation sensitizers, cDNA, siRNA, shRNA, small molecules, and the like. Methods are also provided for using the organoid culture to screen for agents that modulate tissue function.

In some embodiments, the cultures are dissociated after contacting with a therapeutic and/or infectious agent to measure cell-specific changes. In some embodiments, the cells are analyzed or sorted by flow cytometry, e.g. to separate immune cells from lung and stromal elements. The immune cells are optionally further sorted or analyzed by specific markers, e.g. CD45, CD19, CD3, CD4, CD8, CD119, etc., as appropriate to define an immune cell class, such as T cells, B cells, dendritic cells, macrophages, etc. In some embodiments one or more directly or indirectly labeled antibodies specific for an immune cell marker of interest are bound to the population of dissociated cells for sorting or identification by flow cytometry. In some embodiments the dissociation is enzymatic. In some embodiments the enzyme for dissociation is other than trypsin, including dispase collagenase, liberase, etc. In some embodiments the cells are sorted and the population of interest is analyzed for gene expression, as known in the art and including without limitation qRT-PCR. A preamplification step may be performed for about 5 to about 15 cycles, e.g. greater than about 8, about 10, less than about 15, less than about 12 cycles.

In some embodiments, provided are feeder-free, chemically-defined culture of distal human lung organoids, derived from distal lung tissue, and comprising endogenous immune and stromal cells. The cultures may be infected with a respiratory pathogen, e.g. a respiratory virus. Cultures are initiated with fragments of distal lung tissue (“explants”), which are then cultured embedded in a gel substrate that provides an air-liquid interface. Human lung organoids containing epithelial, mesenchymal and immune compartments are grown using a 3D air-liquid interface (ALI) method, derived from human tissue of the distal lung, inclusive of alveolar and terminal bronchiolar cells; and resident immune and stromal cells. These proliferating human ALI organoids strongly contrast with post-mitotic monolayer 2D ALI airway cultures, expanding for greater than 100 days, and contain stroma, e.g. fibroblasts, endothelial cells, etc. as well as diverse immune cells, including T cells, B cells, and macrophages. Human lung ALI cultures can be generated, histologically preserve alveolar air spaces, and retain endogenous immune cells such as T cells, B cells and macrophages. Further, epithelial and immune components express ACE2. The culture medium may be supplemented with an effective dose of factors including, without limitation, one or more of ROCK inhibitors, GSK inhibitors, NOGGIN; EGF; TGF-beta inhibitors; etc., and may include each of these factors.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1B. Characterization of human distal lung ALI organoids. Characterization of human distal lung ALI organoids. a, H&E of human distal human lung ALI organoid, day 15. b, serial brightfield images of one collagen insert of lung ALI organoids, day 1, day 45, and day 100.

FIGS. 2A-2E. Multilineage differentiation in lung ALI organoids. Characterization of epithelial cell types in human lung ALI organoids. Confocal immunofluorescence showing a, club cells (green), b, ciliated cells (white), c, basal cells (green), and d, AT1 (red) and AT2 (green) cells in human lung ALI organoids at day 14. e, confocal immunofluorescence showing AT1 cells (green) and AT2 cells (red) forming cystic structures at day 40.

FIG. 3A-3C. Characterization of immune cells from the human lung ALI organoids. a, confocal immunofluorescence showing tissue resident immune cells (white), epithelial cells (green), and mesenchymal cells (red) in an organoid at day 11. b, FACS plots (gated on live, single cells) staining for surface markers of various immune cells from the human lung ALI organoids, day 12. c, FACS plots (gated on live, single cells) staining for CD68, a macrophage marker, from the human lung ALI organoids, day 13.

FIG. 4A-4C. scRNA-seq of lung organoids. Single-cell RNA sequencing of all cell types in human lung ALI organoids. a, diagram showing schematic of scRNA-seq experiment. b, UMAP of CD45− cells (epithelial and mesenchymal cells) from lung ALI organoid, day 12. c, UMAP of CD45+ cells (immune cells) from lung ALI organoid, day 12.

FIGS. 5A-5B. Lung ALI organoids retain immune cells and stroma. Lung ALI organoids able to retain immune cells and stroma in suspension (apical-out). a, confocal microscopy showing (left) lung ALI organoid apical-in (in collagen), with epithelial, immune, and mesenchymal cells, and (right) interior expression of SARS-CoV-2 receptor ACE2 (red). b, confocal microscopy showing (left) lung ALI organoid apical-out (in suspension) with epithelial, immune, and mesenchymal cells, and (right) exterior expression of SARS-CoV-2 receptor ACE2 (red).

FIGS. 6A-6B. SARS-CoV-2 infection of ALI human lung organoids. SARS-CoV-2 infection of human lung ALI organoids. Confocal microscopy showing SARS-CoV-2 infected cells (green) among epithelial cells (red) and immune cells (white) in a, mock infection, b, 48 hours post-infection.

FIG. 7. SARS-CoV-2 infection of ALI vs. epithelial-only lung organoid.

FIG. 8A-8B. Time course cytokine increase in SARS-CoV-2-infected ALI organoids. Infection of human lung ALI organoids with SARS-CoV-2 leads to time-course dependent increase of cytokines, and is disrupted by remdesivir (RDV). a, qRT-PCR showing fold expression change of cytokines 24 or 48 hours post SARS-CoV-2 infection. b, qRT-PCR showing fold expression change of cytokines 48 hours post SARS-CoV-2 infection with and without remdesivir (RDV).

FIG. 9. Vaccination-dependent adaptive SARS-CoV-2 immune responses in ALI lung organoids. Top. Recombinant spike protein treatment. Bottom. Cytokine qRT-PCR in FACS-sorted organoid CD4+ and CD8+ T cells, stratified by vaccination status. Each dot is an experiment from a distinct human donor.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

The term “culture system” is used herein to refer to the culture conditions in which explants are grown that promote prolonged tissue expansion with proliferation, multilineage differentiation and recapitulation of cellular and tissue ultrastructure.

“Gel substrate”, as used herein has the conventional meaning of a semi-solid extracellular matrix. Gel described here in includes without limitations, collagen gel, matrigel, extracellular matrix proteins, fibronectin, collagen in various combinations with one or more of laminin, entactin (nidogen), fibronectin, and heparin sulfate; human placental extracellular matrix.

An “air-liquid interface” is the interface to which the lung explant cells are exposed to in the cultures described herein. The primary tissue may be mixed with a gel solution, e.g. a collagen gel, which is then poured over a layer of gel formed in a container with a lower semi-permeable support, e.g. a membrane. This container is placed in an outer container that contains the medium such that the gel containing the tissue in not submerged in the medium. The primary tissue is exposed to air from the top and to liquid medium from the bottom, see for example U.S. Pat. No. 9,464,275 herein specifically incorporated by reference.

By “container” is meant a glass, plastic, or metal vessel that can provide an aseptic environment for culturing cells.

The term “sample” with reference to a patient encompasses solid tissue samples such as a biopsy specimen or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like.

The term “explant” is used herein to mean a piece of lung tissue, particularly distal lung tissue and the immune and stromal cells present in that tissue; and the cells thereof originating from the lung tissue that is cultured in vitro, for example according to the methods of the invention. The tissue from which the explant is derived is obtained from an individual.

The term “organoid” is used herein to mean a 3-dimensional growth of lung tissue in culture that retains characteristics of the lung in vivo, e.g. recapitulation of cellular and tissue ultrastructure, immune cell interactions, etc.

As used herein, the term “immune cell” includes cells that are of hematopoietic origin and that play a role in the immune response. Immune cells include lymphocytes, such as B cells and T cells; natural killer cells; dendritic cells; myeloid cells, such as monocytes, macrophages, eosinophils, mast cells, basophils, and granulocytes.

Methods are provided for the culture of small amounts of clinical specimens. Samples of interest include human tissue, e.g. solid lung microbiopsy samples such as needle or fine needle aspirate. Samples may be taken at a single timepoint, or may be taken at multiple timepoints. Samples may be as small as 10⁷ cells, 10⁶ cells, 10⁵ cells, or less.

The phrase “mammalian cells” means cells originating from mammalian tissue. Typically, in the methods of the invention pieces of tissue are obtained surgically, e.g. biopsy, needle biopsy, etc. and minced to a size less than about 1 mm³, and may be less than about 0.5 mm³, or less than about 0.1 mm³. “Mammalian” used herein includes human, equine, bovine, porcine, canine, feline, rodent, e.g. mice, rats, hamster, primate, etc. “Mammalian tissue cells” and “primary cells” have been used interchangeably.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

The term “candidate cells” refers to any type of cell that can be placed in co-culture with the tissue explants described herein. Candidate cells include without limitations, genetically engineered T cells including without limitation CAR-T cells, dendritic cells, phagocytic cells T cells, B cells, etc.

The term “candidate agent” means any oligonucleotide, polynucleotide, siRNA, shRNA, gene, gene product, peptide, antibody, small molecule or pharmacological compound that is introduced to an explant culture and the cells thereof as described herein to assay for its effect on the explants.

The term “contacting” refers to the placing of candidate cells or candidate agents into the explant culture as described herein. Contacting also encompasses co-culture of candidate cells with tissue explants for at least 1 hour, or more than 2 hrs or more than 4 hrs in culture medium prior to placing the tissue explants in a semi-permeable substrate. Alternatively, contacting refers to injection of candidate cells into the explant, e.g. into the lumen of an explant.

“Screening” refers to the process of either co-culturing candidate cells with or adding candidate agents to the explant culture described herein and assessing the effect of the candidate cells or candidate agents on the explant, including without limitation immune cells present in the explant. The effect may be assessed by assessing any convenient parameter, e.g. phenotypic changes, protein expression, mRNA expression, etc.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of infection in a mammal, particularly in a human, and includes one or more of: (a) preventing disease; (b) inhibiting the disease, i.e., arresting its development; and (c) relieving the disease or its symptoms, i.e., causing regression of the disease or its symptoms. Treating may also refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize infection and the sequelae of infection. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of one or more therapeutic agents; or to the screening of two or more agents in a culture. When administered in combination, each component can be administered at the same time or sequentially in any order at different points in time. Thus, each component can be administered separately but sufficiently closely in time so as to provide the desired therapeutic effect.

“Concomitant administration” means administration of one or more components at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

As used herein, the term “infection” refers to any state in at least one cell of an organism (i.e., a subject) is infected by an infectious agent. As used herein, the term “infectious agent” refers to a foreign biological entity, i.e. a pathogen. Infectious agents include, but are not limited to bacteria, viruses, protozoans, and fungi. Infectious diseases are disorders caused by infectious agents. Some infectious agents cause no recognizable symptoms or disease under certain conditions, but have the potential to cause symptoms or disease under changed conditions.

Pathogens of interest include respiratory pathogens, e.g. viruses, bacteria and protozooans that infect the lungs, particularly in lower respiratory infections. Pathogens of interest include, for example, influenza, rhinovirus, adenovirus, coronavirus such as SARS-CoV1, SARS-CoV2, MERS-CoV, etc.; tuberculosis, etc. Respiratory viruses are a frequent causative agents of disease in humans, with significant impact on morbidity and mortality worldwide, mainly in children. A number of human respiratory viruses circulate commonly in all age groups and are recognized as adapted to efficient person-to-person transmission. And respiratory pathogen can be used to infect a culture of the disclosure, and can be studied by monitoring and screening methods of the disclosure. Common respiratory viruses include influenza: ADV, adenovirus; HBoV, human bocavirus; HCoV, human coronavirus including SARS-CoV1 and SARS-CoV2; HMPV, human metapneumovirus; HPIV, human parainfluenza virus; HRSV, human respiratory syncytial virus; HRV, human rhinovirus, etc. which can be studied with the cultures of the disclosure.

Influenza virus. There are four types of influenza viruses, A, B, C, and D. Influenza types A and B cause human infection annually during the epidemic season. Influenza A has several subtypes according to the combination of hemagglutinin (H) and the neuraminidase (N) proteins that are expressed on the surface of the viruses. Influenza viruses have a negative-sense, single-stranded RNA genome that is segmented. There are 18 different hemagglutinin subtypes and 11 different neuraminidase subtypes (H1-18 and N1-11). Influenza A viruses can be characterized by the H and N types such as H1N1 and H3N2. Influenza B viruses are classified into lineages and strains. Influenza viruses replicate in the epithelial cell lining of the upper and lower respiratory tracts. Illness during infection is primarily the result of lung inflammation and compromise caused by epithelial cell infection and death, combined with inflammation caused by the immune system's response to infection. Severe respiratory illness can be caused by multiple, non-exclusive mechanisms, including obstruction of the airways, loss of alveolar structure, loss of lung epithelial integrity due to epithelial cell infection and death, and degradation of the extracellular matrix that maintains lung structure. In particular, alveolar cell infection appears to drive severe symptoms since this results in impaired gas exchange and enables viruses to infect endothelial cells, which produce large quantities of pro-inflammatory cytokines. Pneumonia caused by influenza viruses is characterized by high levels of viral replication in the lower respiratory tract, accompanied by a strong pro-inflammatory response

Human respiratory syncytial virus (HRSV) virions are heterogeneous in size and shape, and consist of a helical nucleocapsid containing a negative-sense single-stranded RNA, tightly bound to the nucleoprotein (N). Two HRSV groups, A and B, were originally distinguished based on the antigenic differences in the attachment glycoprotein G. HRSV is the single most frequent cause of lower respiratory tract infection (LRTI) leading to morbidity and mortality in children worldwide. HRSV replicates in respiratory epithelia, reaching high titers in nasal secretions and causing virus shedding for up to 3 weeks after the end of symptoms. Cell-to-cell spread leads to involvement of the entire respiratory tree by HRSV, reaching bronchioles 1-3 days after the onset of symptoms, inducing necrosis of ciliated cells, syncytia formation, peribronchiolar inflammation with abundant lymphocytes and macrophages, and impairment of secretion clearance, resulting in small airway obstruction and lung hyperinflation typical of bronchiolitis. HRSV pneumonia with interstitial mononuclear infiltrate, eosinophilic cytoplasmic inclusions in epithelial cells, and multinucleated giant cells, frequently coexist with bronchiolitis. HRSV disease is particularly severe in young babies, whose immature airways are unable to compensate for the virus-induced damage.

Human parainfluenza viruses (HPIVs) are frequent causes of LRTIs in infants and children worldwide. HPIVs share structural and biological characteristics with HRSV and are distributed in two genera of the family Paramyxoviridae. HPIVs are classified antigenically into types 1-4, and HPIV-4 has subtypes A and B. The virions are pleomorphic, with single-stranded negative-sense RNA, ranging from 150 to 200 nm in diameter. The virus does not persist long in the environment and is transmitted mainly by large droplets and fomites. HPIV replicates in ciliated cells causing cytolysis of the respiratory mucosa. The infection begins in the upper respiratory tract and disseminates down the respiratory tree. The larynx and trachea are mostly involved in the croup syndrome, and extensive involvement of the lower respiratory tree may be present in tracheobronchitis, bronchopneumonia, and bronchiolitis. Similar to what occurs with HRSV, amplified inflammatory response induced by viral infection of epithelial cells causes mononuclear interstitial infiltrate, epithelial necrosis, inflammatory exudate into the alveoli, and hyaline membrane formation in the lungs. In cases of croup, mononuclear inflammatory cell infiltrate is seen in the subglotic area.

Human metapneumovirus (HMPV) is a frequent cause of community-acquired ARI in children and adults worldwide. HMPV particles are enveloped, pleomorphic, spherical, or filamentous particles, of about 209 nm in diameter. Like other paramyxoviruses, HMPV has a negative-sense, single-stranded RNA genome, and the viral replication occurs in a gradient manner. Little is known about HMPV-specific mechanisms of pathogenesis. Animal studies show disruption of the respiratory epithelium, epithelial cell sloughing, and inflammatory infiltrates in the lung. In pathologic studies of humans with underlying diseases and HMPV infection, the main findings are acute and organizing lung injury, diffuse alveolar damage, sloughed epithelial cells with eosinophilic cytoplasmic inclusions, multinucleated giant cells, histiocytes, and hyaline membrane formation.

Human rhinoviruses (HRVs) are the most frequent respiratory pathogens of humans, and the most commonly detected viruses in samples from common cold sufferers. HRVs are small, nonenveloped, positive-stranded RNA viruses in the family Picornaviridae, genus Rhinovirus, distributed in two species, A (75 serotypes) and B (25 serotypes). HRV replication is restricted to the respiratory epithelium, taking place in scattered ciliated cells of the nose and in nonciliated cells of the nasopharynx, and this tropism seems to be a consequence of receptor availability. Infection of a limited number of cells triggers the nuclear translocation of NF-κB and gene expression of cytokines, chemokines, and inflammatory mediators. These, in association with the stimulation of local parasympathetic nerve endings, result in the development of cold symptoms. Kinins, prostaglandins, proinflammatory cytokines, and chemokines may contribute to vasodilation, increased vascular permeability, influx of polymorphonuclear leukocytes, exocrine gland secretion, and nerve ending stimulation, resulting in nasal obstruction, rhinorrhea, sneezing, cough, and sore throat.

Adenoviruses are nonenveloped, icosahedral DNA viruses of the genus Mastadenovirus, family Adenoviridae. Respiratory infections by adenoviruses occur worldwide and with no apparent seasonality. Symptomatic infections may involve all parts of the respiratory tract and generally initiate in the upper respiratory epithelium. Adenovirus infection results in necrosis of cells of airway epithelia and may cause viremia by systemic virus dissemination in immunocompromised persons. Bronchiolitis, interstitial pneumonitis, and mononuclear cell infiltrates are part of the inflammatory process in the lungs. In addition to lytic infection, adenoviruses may become latent in epithelial and lymphoid cells, which is probably important to maintain the virus in populations.

Human coronavirus (HCoV) subtypes 229E and OC43 were the only coronaviruses identified in humans until 2003, when identification of the severe acute respiratory syndrome (SARS) coronavirus led to a renewed research on HCoVs. Coronaviruses are enveloped viruses with distinct virion morphology, displaying widely spaced, long petal-shaped spikes at the surface, that confer to the virus a crownlike appearance, origin of the name corona. The viral envelope contains a long helical nucleocapsid with single-, positive-stranded. HCoVs have been found throughout the world and are considered to be the second most frequent cause of common colds. HCoV-229E and -OC43 cause common colds with variable frequency.

SARS-CoV-2 is a virus of the species severe acute respiratory syndrome-related coronavirus (SARSr-CoV), related to the SARS-CoV-1 virus. It is of zoonotic origins and has close genetic similarity to bat coronaviruses. SARS-CoV-2 is a member of the subgenus Sarbecovirus (beta-CoV lineage B). Coronaviruses undergo frequent recombination. There are many thousands of variants of SARS-CoV-2, which can be grouped into the much larger clades. The mechanism of recombination in unsegmented RNA viruses such as SARS-CoV-2 is generally by copy-choice replication, in which gene material switches from one RNA template molecule to another during replication. SARS-CoV-2 RNA sequence is approximately 30,000 bases in length. A distinguishing feature of SARS-CoV-2 is its incorporation of a polybasic site cleaved by furin, which appears to be an important element enhancing its virulence. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. The spike protein is the protein responsible for allowing the virus to attach to and fuse with the membrane of a host cell; specifically, its S1 subunit catalyzes attachment, the S2 subunit fusion. Initial spike protein priming by transmembrane protease, serine 2 (TMPRSS2) is essential for entry of SARS-CoV-2. After a SARS-CoV-2 virion attaches to a target cell, the cell's TMPRSS2 cuts open the spike protein of the virus, exposing a fusion peptide in the S2 subunit, and the host receptor ACE2. After fusion, an endosome forms around the virion, separating it from the rest of the host cell. The virion escapes when the pH of the endosome drops or when cathepsin, a host cysteine protease, cleaves it. The virion then releases RNA into the cell.

It is common to observe a dysregulation of the innate immune responses in COVID-19 patients. Upregulated IL-6, potentially produced by M$ or monocytes, has been reported in COVID-19 patients in multiple studies. Although an activated innate immune response was observed in SARS-CoV-2-infected patients, their immune system failed to launch robust type I and type III IFN responses. The expression of the SARS-CoV-2 receptor ACE2 on the surface of Mϕ may contribute to the pathogenicity of COVID-19. Antibody responses can be detected as early as the first-week post symptom onset, and most patients show antibody responses within 2 weeks after symptom onset. T cell reactivity against SARS-CoV-2 can be detected about 1-week post symptom onset, with specific T cell reactions detected against the S, M, NP as well as non-structural proteins. Peripheral CD4+ and CD8+ T cell depletion has been observed in COVID-19 patients and especially in severe cases. Of note, PD-1 is upregulated in both CD4+ and CD8+ T cell subsets in severe patients. A potential cause of the T cell exhaustion could be the inflammatory cytokines, such as IL-6, which is known to induce T cell exhaustion.

Human bocavirus (HBoV) is a novel member of the family Parvoviridae, provisionally classified by sequence homology and genome organization in the genus Bocavirus, which already included two other viruses: the bovine parvovirus 1 and canine minute virus. HBoV virions consist of small nonenveloped icosahedral particles with a single-stranded DNA genome of approximately 5300 nt, organized in a way similar to that of other known bocaviruses, with three ORFs, two encoding the nonstructural proteins NS1 and NP-1, and a third nesting the two capsid proteins, VP1 and VP2. HBoV circulates worldwide and prevalence studies, done mostly by PCR in respiratory samples from children, have shown detection rates of 1.5-19%, depending on the regions where studies were conducted and the sensitivity of the assays. The sites of HBoV replication have not been determined, but the virus is apparently not confined to the respiratory tract, since it has been detected in stools and serum, suggesting systemic infection.

Bacterial and protozoan pathogens are less common than viral but can include Mycobacterium tuberculosis, Streptococcus pneumoniae, Mycoplasma pneumoniae, Haemophilus influenzae, Chlamydophila pneumoniae; Chlamydia psittaci; Coxiella burnetiid; Legionella pneumophila Staphylococcus aureus; Klebsiella pneumoniae; cryptosporidiosis can cause outbreaks and sporadic cases of respiratory illness.

### Cultures

Culture systems and methods are provided for culture of solid lungs, including stromal and immune cells associated with the lungs in vivo. The cultures can be maintained for up to 5 days, up to 7 days, up to 10 days, up to 15 days, up to 21 days, up to 28 days, up to 100 days, up to 180 days, or more. Cultures for the study of immune components may be used in shorter term cultures, e.g. up to about 28 days, or can be supplemented with exogenous cytokines. In some embodiments, tissue, i.e. distal lung tissue, is obtained from any mammalian species, e.g. human, equine, bovine, porcine, canine, feline, rodent, e.g. mice, rats, hamster, primate, etc.

Lung tissue may be obtained by any convenient method, e.g. by biopsy, during surgery, by needle, etc., and is typically obtained as aseptically as possible. Upon removal, tissue can be immersed in ice-cold buffered solution, e.g. PBS, Ham's F12, MEM, culture medium, etc. Pieces of tissue may be minced to a size less than about 1 mm³, and may be less than about 0.5 mm³, or less than about 0.1 mm³. The minced tissue is mixed with a gel substrate, e.g. a collagen gel solution, e.g. collagen; a matrigel solution, etc. Subsequently, the tissue-containing gel substrate is layered over a layer of gel (a “foundation layer”) in a container with a lower semi-permeable support, e.g. a membrane, supporting the foundation gel layer, and the tissue-containing gel substrate is allowed to solidify. This container is placed into an outer container containing a suitable medium, that may be supplemented with fetal calf serum (FCS) at a concentration of from about 1 to about 25%, usually from about 5 to about 20%, etc.

The arrangement described above allows nutrients to travel from the bottom, through the membrane and the foundation gel layer to the gel layer containing the tissue. The level of the medium is maintained such that the top part of the gel, i.e. the gel layer containing the explants, is not submerged in liquid but is exposed to air. Thus the tissue is grown in a gel with an air-liquid interface. A description of an example of an air-liquid interface culture system is provided in Ootani et al. in Nat Med. 2009 June; 15(6):701-6, the disclosure of which is incorporated herein in its entirety by reference. The air-liquid interface organoid cultures could be moved into other formats such as multi-wells for screening or in submerged 2D or 3D geometries where the cells are placed underneath the tissue culture medium.

In some embodiments the organoids have everted polarity. 3D basal and alveolar organoids are typically oriented with the basolateral surface oriented outwards, i.e. facing the extracellular matrix substratum, which can hinder infection of the apical ACE2-expressing luminal surface. The cultures can be everted by removal from extracellular matrix gel and growth in suspension, robustly generating organoids with their apical surfaces oriented outward. Within from about 1 to about 3 days, around about 2 days, non-polarized organoids reorganize into apical-out epithelial spheroids with microvilli, apical junctions, and some motile cilia facing the organoid exterior. Everted organoids display outwardly facing club cells with apical secretory granules. SARS-CoV-2 readily infected apical-out mixed distal lung organoids, with direct SARS-CoV-2 infection of AT2 cells, and club cells as a novel target population.

The medium for culture may comprise an effective dose of an EGF agent, e.g. human EGF protein, agonist antibodies, etc.; and an effective dose of a BMP antagonist, including without limitation, NOGGIN. The medium may comprise extracellular matrix, unless it is an everted culture. The medium may comprise an inhibitor of TGF-β; a ROCK inhibitor, e.g. Y-27632; GSK inhibitor; etc.

As used herein, the term “BMP” refers to the family of bone morphogenetic proteins, which reference sequence may be found in Genbank, for example BMP-2 accession number NP_001191. Antagonists include antibodies and fragments thereof that block activity of the cognate BMP receptor. Inhibitors (antagonists) of the BMP pathway include but are not limited to, e.g., NOGGIN, CHORDIN, LDN-193189 (4-[6-[4-(1-Piperazinyl)phenyl]pyrazolo[1,5-a]pyrimidin-3-yl]-quinoline hydrochloride), DMH1 (4-[6-[4-(1-Methylethoxy)phenyl]pyrazolo[1,5-a]pyrimidin-3-yl]-quinoline), Dorsomorphin (6-[4-[2-(1-Piperidinyl)ethoxy]phenyl]-3-(4-pyridinyl)-pyrazolo[1,5-a]pyrimidine dihydrochloride), K 02288 (3-[(6-Amino-5-(3,4,5-trimethoxyphenyl)-3-pyridinyl]phenol), ML 347 (5-[6-(4-Methoxyphenyl)pyrazolo[1,5-a]pyrimidin-3-yl]quinoline), DMH-1, antibodies to BMPs and BMP receptors, BMP inhibitory nucleic acids, and the like. In some instances, the agents, as described above include, e.g., those that are commercially available, e.g., from such suppliers such as Tocris Bioscience (Bristol, UK), Sigma-Aldrich (St. Louis, MO), Santa Cruz Biotechnology (Santa Cruz, CA), and the like.

Inhibitors of the TGF-beta pathway include but are not limited to, e.g., A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids, and the like.

The inhibitor of GSK-3p may be a small molecule chemical compound (e.g., TWS119, BIO, CHIR-99021, SB 216763, SB 415286, CHIR-98014 and the like).

Exogenous cytokines can be added to the medium to provide support for immune cell populations. Typically IL-2 is used to support T cells, and can be use in combination with CD3/CD28 antibodies immobilized on beads. Other cytokines useful for T cell culture include, without limitation, IL-4, IL-7, IL-15, etc. B cells can also be supported with IL-2, but may further benefit from one or both of IL-21 and BAFF. Addition cytokines useful for B cell support include IL-6, IL-10, etc.

The lung tissue cell suspension may be contacted with agents by any convenient means. Generally the agents are added to culture media, as described herein, within which cells of the instant disclosure are grown or maintained, such that the agent is present, in contact with the cells, at an effective concentration to produce the desired effect.

The effective concentration of an agent will vary and will depend on the agent. In addition, in some instances, the effective concentration may also depend on the cells being induced, the culture condition of the cells, other agents co-present in the culture media, etc. As such, the effective concentration of agents may range from 1 ng/mL to 10 μg/mL or more, including but not limited to, e.g., 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 1-5 ng/mL, 1-10 ng/mL, 1-20 ng/mL, 1-30 ng/mL, 1-40 ng/mL, 1-50 ng/mL, 5-10 ng/mL, 5-20 ng/mL, 10-20 ng/mL, 10-30 ng/mL, 10-40 ng/mL, 10-50 ng/mL, 20-30 ng/mL, 20-40 ng/mL, 20-50 ng/mL, 30-40 ng/mL, 30-50 ng/mL, 40-50 ng/mL, 1-100 ng/mL, 50-100 ng/mL, 60-100 ng/mL, 70-100 ng/mL, 80-100 ng/mL, 90-100 ng/mL, 10-100 ng/mL, 50-200 ng/mL, 100-200 ng/mL, 50-300 ng/mL, 100-300 ng/mL, 200-300 ng/mL, 50-400 ng/mL, 100-400 ng/mL, 200-400 ng/mL, 300-400 ng/mL, 50-500 ng/mL, 100-500 ng/mL, 200-500 ng/mL, 300-500 ng/mL, 400 to 500 ng/mL, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 μg/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5-9 μg/mL, 0.5-10 μg/mL, and the like.

The continued growth of the explant may be confirmed by any convenient method, e.g. phase contrast microscopy, stereomicroscopy, histology, immunohistochemistry, electron microscopy, etc. In some instances, cellular ultrastructure and multi-lineage differentiation may be assessed. Ultrastructure of the intestinal explants in culture can be determined by performing Hematoxylin-eosin staining, PCNA staining, electron microscopy, and the like using methods known in the art.

Methods of analysis include, without limitation, single cell RNA sequencing of epithelial and immune components, microscopy, including fluorescence microscopy and staining, H&E and 3D confocal imaging with staining for differentiation and polarity markers; quantitative RT-PCR, e.g. to measure the accumulation of viral genomes or mRNA after infection; spatial imaging analysis of stained markers in FFPE sections at single cell resolution, and the like. The viability of lung epithelial and immune cells can be monitored throughout the infection process, e.g. by annexin V staining, etc.

A feature of the culture is the diversity of cells present, which include, without limitation E-Cadherin+ epithelial cells closely associated with Vimentin+ mesenchymal cells and interspersed with CD45+ immune cells. CD31 staining revealed endothelial cells forming network-like structures within the organoids. SFTPC+AT2 cells grew in distinct clusters within the organoids and remained proliferative with KI67+ staining for over 60 day. Similar to the AT2 cells, KRT5+ basal cells also grew in distinct clusters, forming structures that resemble bronchioles extending into alveoli. AT2 cells and basal cells were seen within the same large organoid, contrasting with epithelial-only spheroids in which basal and alveolar organoids were only seen in mutually exclusive organoids. Additionally, the organoids contained rare SCGB1A1+ club cells and acetylated-tubulin+ ciliated cells. Notably, the ciliated cells only appeared after about three weeks in culture. The organoids also contained alveoli-like cystic structures lined with HT1-56+AT1 cells.

There is also a diversity of immune cells. Earlier timepoints consisted mostly of lymphocytes, with CD4 T cells being most abundant followed by CD8 T cells and relatively rare B cells. Without exogenous cytokine support lymphocyte populations decreased substantially over time, with virtually none remaining at day 33 and beyond. SSC-hi, CD68+, CD11c− macrophages began as a minority of total CD45+ cells but persisted over time and eventually comprised the majority of CD45+ cells. Additionally, there was a small population of CD3E+, CD8−, NKG7+NK-T cells, CD3E, CD4, FOXP3 positive Tregs and SDC1+ plasma cells.

Experimental modifications may be made by any method known in the art, for example, as described below with regard to methods for providing candidate agents that are nucleic acids, polypeptides, small molecules, viruses, etc. to explants and the cells thereof for screening purposes. In particular, infection of the tissue is of interest, and screening of infected tissue with candidate therapeutic agents.

### Utility

Organoids prepared by the subject methods may be used in basic research, e.g. to better understand the basis of disease and infection, and in drug discovery, e.g. as reagents in screens such as those described further below, and for diagnostic purposes. Organoids are also useful for assessing the pharmacokinetics and pharmacodynamics of an agent, e.g. the ability of a mammalian tissue to absorb an active agent, the cytotoxicity of agents on primary mammalian tissue, etc. The immune component of these organoids are useful for assessing vaccines, adjuvants, or identifying the effects of candidate therapeutics on disease or infection-related immune responses.

### Screening Methods

In some aspects of the invention, methods and culture systems are provided for screening candidate agents or cells for an activity of interest. In these methods, candidate agents or cells are screened for their effect on cells in the organoids of the invention. Organoids of interest include those comprising unmodified cells, those comprising experimentally modified cells, and particularly infected cells. In some embodiments the organoid is obtained from an individual that has been vaccinated against a virus of interest.

“Screening” refers to the process of either co-culturing candidate cells with or adding candidate agents to the explant culture described herein and assessing the effect of the candidate cells or candidate agents on the explant. The effect may be assessed by assessing any convenient parameter, e.g. the production of cytokines, the expression of genes of interest by one or more of the cells types, the ultrastructure of the explant, etc. The effect of candidate cells, viruses, bacteria, candidate agents, etc. on the explant can be further evaluated by single cell RNA sequencing of epithelial and immune components, microscopy, including fluorescence microscopy and staining, H&E and 3D confocal imaging with staining for differentiation and polarity markers; quantitative RT-PCR, e.g. to measure the accumulation of viral genomes or mRNA after infection; spatial imaging analysis of stained markers in FFPE sections at single cell resolution, and the like. The viability of lung epithelial and immune cells can be monitored throughout the infection process, e.g. by annexin V staining, etc.

In some embodiments, the cells in the cultured explants may be experimentally modified. For example, the explant cells may be modified by exposure to viral or bacterial pathogens, e.g. to develop a reagent for experiments to assess the anti-viral or anti-bacterial effects of therapeutic agents. The explant cells may be modified by altering patterns of gene expression, e.g. by providing reprogramming factors to induce pluripotency or otherwise alter differentiation potential, or to determine the effect of a gain or loss of gene activity on the ability of cells to form an explant culture. The explant cells may be modified such that they are transformed with growth factors or cytokines or other genes to modulate viral infection phenotypes on immune cells or intestinal epithelial cells.

Experimental modifications may be made by any method known in the art, for example, as described below with regard to methods for providing candidate agents that are nucleic acids, polypeptides, small molecules, viruses, etc. to explants and the cells thereof for screening purposes.

The effect of an agent or cells is determined by adding the agent or cells to the cells of the cultured explants as described herein, usually in conjunction with a control culture of cells lacking the agent or cells. The effect of the candidate agent or cell is then assessed by monitoring one or more output parameters. Parameters are quantifiable components of explants or the cells thereof, particularly components that can be accurately measured, in some instances in a high throughput system. For example, a parameter of the explant may be the growth, differentiation, gene expression, proteome, phenotype with respect to markers etc. of the explant or the cells thereof, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

In some embodiments, candidate agent, pathogen, or cells are added to the cells within the intact organoid. In other embodiments, the organoids are dissociated, and candidate agent, pathogen, or cells is added to the dissociated cells. The cells may be freshly isolated, cultured, genetically altered as described above; or the like. The cells may be environmentally induced variants of clonal cultures: e.g. split into independent cultures and grown into organoids under distinct conditions, for example with or without pathogen; in the presence or absence of other cytokines or combinations thereof. The manner in which cells respond to an agent, particularly a pharmacologic agent, including the timing of responses, is an important reflection of the physiologic state of the cell.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Therapeutic agents are of interest for screening, for example comparing and including currently available drugs, e.g. anti-viral drugs such as Tamiflu, remdesavir, darunavir, nelfinavir, and novel therapeutics, e.g. anti-pathogen mAb, etc. Antagonists of immune activation, e.g. anti-IL6R, Kevzara, Actemra to model blunting of the inflammatory response and to measure inhibition of lung epithelial damage is of interest.

Additional candidate agents include vaccines, with or without adjuvants, which can be tested in this system to assess induction of immune responses.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

The polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of explant or cell samples, usually in conjunction with explants not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the explant, e.g. into the lumen of the explant, and their effect compared to injection of controls.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

Screens for agents to prevent, treat or vaccinate against a disease. Other examples of screening methods of interest include methods of screening a candidate agent for an activity in treating or preventing a disease. The candidate agent can also be a vaccine with or without adjuvants. In such embodiments, the explant models the disease, e.g. the explant may have been obtained from a diseased tissue, or may be experimentally modified to model the disease by, e.g., genetic mutation. Parameters such as explant growth, cell viability, cell ultrastructure, tissue ultrastructure, etc. find particular use as output parameters in such screens.

Screens to determine the pharmacokinetics and pharmacodynamics of agents. Other examples include methods of screening a candidate agent for toxicity to tissue. In these applications, the cultured explant is exposed to the candidate agent or the vehicle and its growth and viability is assessed. In these applications, analysis of the ultrastructure of the explants is also useful.

### High Throughput Screens

In some aspects of the invention, methods and culture systems are provided for screening candidate agents in a high-throughput format. By “high-throughput” or “HT”, it is meant the screening of large numbers of candidate agents or candidate cells simultaneously for an activity of interest. By large numbers, it is meant screening 20 more or candidates at a time, e.g. 40 or more candidates, e.g. 100 or more candidates, 200 or more candidates, 500 or more candidates, or 1000 candidates or more.

In some embodiments, the high throughput screen will be formatted based upon the numbers of wells of the tissue culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed. High throughput screens formatted in this way may be achieved by using, for example, transwell inserts. Transwell inserts are wells with permeable supports, e.g. microporous membranes, that are designed to fit inside the wells of a multi-well tissue culture dish. In some instances, the transwells are used individual. In some instances, the transwells are mounted in special holders to allow for automation and ease of handling of multiple transwells at one time.

To achieve the numbers of organoids necessary to perform a high-throughput screen, a primary organoid (that is, an organoid that has been cultured directly from tissue fragments) is dissociated into a single cell suspension and replated across multiple transwells to generate secondary organoids in a multiwell format. Dissociation may be by any convenient method, e.g. manual treatment (trituration), or chemical or enzymatic treatment with, e.g. EDTA, trypsin, papain, etc. that promotes dissociation of cells in tissue. The dissociated organoid cells are then replated in transwells at a density of 10,000 or more cells per 96-well transwell, e.g. 20,000 cells or more, 30,000 cells or more, 40,000 cells or more, or 50,000 cells or more. Additional iterations of dissociation and plating may be performed to achieve the desired numbers samples of organoids to be treated with agent.

In some embodiments, the secondary (or tertiary, etc.) organoids may be cultured first, after which candidate agents or cells are added to the organoid cultures and parameters reflective if a desired activity are assessed. In other embodiments, the candidate agents or cells are added to the dissociated cells at replating. This latter paradigm may be particularly useful for example for assessing candidate agents/cells for an activity that impacts the differentiation of cells of the developing organoid. Any one or more of these steps may be automated as convenient, e.g. robotic liquid handling for the plating of explants, addition of medium, and/or addition of candidate agents; robotic detection of parameters and data acquisition; etc.

The following examples are put forth so as to provide those of ordinary skill in the art with a complete disclosure and description of how to make and use the subject invention, and are not intended to limit the scope of what is regarded as the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Human distal lung culture: Human distal lung was washed with PBS, minced finely on ice, and resuspended in Cultrex Rat Collagen I ((R&D). 1 mL of tissue-collagen suspension was layered on top of pre-solidified 1 mL collagen gel within a 30 mm, 0.4 mm inner transwell. After fully solidifying, the 6 collagen transwells were placed in a standard 6-well tissue culture plate. 1 mL of lung ALI culture media (see below) was added into the tissue culture plate, below the bottom surface of the collagen-containing transwell. Media was changed 2×/week.

Lung ALI culture media: Advanced DMEM/F12 (Invitrogen) supplemented with 10 mM nicotinamide, n-acetyl cysteine, 1×B27 supplement minus vitamin A, recombinant human NOGGIN (100 ng/mL, R&D Systems), recombinant human EGF (50 ng/mL, R&D Systems), and TGF-beta inhibitor A83-01 (100 nM, Tocris). This mixture was then supplemented with 10% fetal bovine serum, Y-27632 (Peprotech), CHIR 99021 (R&D).

Histologic analysis of ALI organoids: Collagen from transwell containing ALI organoids were fixed in 10% formalin for 1 hr at room temperature, cut into thin slices, and placed into a histology cassette with 70% ethanol. The collagen was then paraffin embedded and sectioned (4-5 mm). Sections were deparaffinized and stained with H&E for histological analysis.

Serial brightfield imaging: Tissue culture plates containing the ALI organoids in transwells were imaged serially with a Keyence microscope. Images were stitched using BZ-Wide viewer software.

Whole-mount staining and confocal microscopy of ALI organoids: collagen containing ALI organoids was cut away from the transwell and fixed in 4% PFA for 1 hour at room temp. PFA was neutralized with 1×PBS-glycine for 30 min at room temp, then blocked and permeabilized with 10% donkey serum in 1×IF buffer for 2 hours at room temp. Organoids were then stained with primary antibodies at room temp for 3 days overnight, followed by 3×30 min washes with 1×IF wash buffer. Secondary staining was carried out with 1:1000 fluorescent donkey secondaries (Jackson Immunoresearch) and DAPI for 4 hours at room temp, then washed 3×30 min. Organoids were then mounted on slides with mounting buffer (Prolong Gold Antifade mounting media, Thermo). Images were acquired using Zeiss LSM-900 confocal microscope, and viewed in 3-D using Imaris software.

Staining buffer recipes:

### 10×PBS:Glycine (500 mL):

### 38.00 g NaCl

9.38 g Na₂HPO₄
2.07 g NaH₂PO₄

### 37.50 g Glycine

Fill to 500 mL with 1×PBS
pH 7.4 and filter sterilize
10×IF-permeabilization buffer (500 mL)

### 38.00 g NaCl

9.38 g Na₂HPO₄
2.07 g NaH₂PO₄

### 2.5 g NaN₃

### 5.0 g BSA (Fraction V)

### 10.00 mL Triton X-100

2.5 mL tween-20
Fill to 500 mL with 1×PBS
pH 7.4 and filter sterilize

FACS analysis of immune surface markers from lung ALI organoids: Collagen containing lung ALI organoids was digested with Collagenase Type 4 (Worthington), for 30 min with shaking at 37 degrees C., then centrifuged and washed with media containing FBS to quench collagenase. Organoids (now dissociated from collagen) were digested to single-cell with Liberase TL (Sigma) and DNAse (Worthington) for 30 min with shaking at 37 degrees C., and washed and quenched with FACS buffer (5 mM EDTA+5% FBS). Cells were stained for viability with Zombie Aqua (Biolegend) 1:500 in FACS buffer for 20 min on ice, protected from light. After washing with FACS buffer, cells were stained for surface markers with antibodies listed below, all at 1:100. Compensation was performed using OneComp eBeads™ Compensation Beads (Thermo) and primary antibodies 1:400. Sorting and analysis was performed on a BD FACSAria II SORP, with further data analysis in FlowJo.

Eversion of human lung ALI organoids: Lung ALI organoids were grown as previously described in collagen for 5-10 days. To evert, collagen was removed using Collagenase Type 4 (Worthington) for 30 min with shaking at 37 degrees C. Collagenase was washed and quenched with FBS containing media 3×, for 10 min each at room temp. Organoids were collected by centrifuging at 100×g for 3 min at room temp. They were then resuspended in the lung ALI media (previously described) and plated in 1.5-2 mL each in a low-attachment 6-well plate (Corning), and allowed to restructure for 2-5 days.

Single-cell RNA sequencing of human lung ALI organoids: Organoids were harvested either from collagen or suspension, digested into single-cell as previously described, and sorted on a BD FACSAria II SORP for singlet discrimination, followed by live/dead gating, followed by CD45−/+ gating. Cellular suspensions were loaded on a Chromium Single Cell Controller instrument (10× Genomics, Pleasanton, CA, USA) to generate single-cell GEMs. Libraries for sequencing was prepped as per manufacturer's instructions using a 5′ library prep kit (10× Genomics).

SARS-CoV-2 infection of lung ALI organoids in suspension: Human lung ALI organoids were grown in collagen for 5-10 days, put into suspension for 2-5 days, counted, then infected with SARS-CoV-2 prior to day 14 in culture. Organoids were resuspended in virus media or an equal volume of mock media, at a MOI of 1 relative to total organoid cells in the sample, and then incubated at 37° C. under 5% CO₂ for 2 hours. Organoids were then plated in suspension in lung ALI organoid media. At the indicated timepoints, organoids were washed with lung organoid media and PBS and either resuspended in TRIzol LS (Thermo Fisher), freshly-made 4% PFA in PBS. All SARS-CoV-2 work was performed in a class II biosafety cabinet under BSL3 conditions at Stanford University. For remdesivir experiments (RDV), organoids were infected with SARS-CoV-2 as described above, and spiked with 10 uM RDV at 24 hours post-infection.

qPCR analysis of SARS-CoV-2 RNA. RNA from SARS-CoV-2-infected organoids was extracted by adding 750 μl TRIzol (Thermo Fisher Scientific), and purified using an RNA Clean & Concentrator-25 kit (Zymo Research) as per manufacturer instructions. All RNA samples were treated with DNase (Turbo DNA-free kit, Thermo Fisher Scientific). The Brilliant II SYBR Green QRT-PCR 1-Step Master Mix (VWR) was used to convert RNA to cDNA and amplify specific RNA regions on the CFX96 Touch real-time PCR detection system (Bio-Rad). RT reaction was performed for 30 min at 50° C., 10 min at 95° C., followed by two-step qPCR with 95° C. for 10 seconds and 55° C. for 30 seconds, for a total of 40 cycles. Primer sequences are as follows:

| Gene | Forward primer | Reverse primer |
|---|---|---|
| TNF-alpha | TCTTCTCGAACCCCGAGTGA | CCTCTGATGGCACCACCAG |
| IL-6 | GTAGCCGCCCCACACAGACAGCC | GCCATCTTTGGAAGGTTC |
| M-CSF | GGAGACCTCGTGCCAAATTA | TATCTCTGAAGCGCATGGTG |
| 18S | GGCCCTGTAATTGGAATGAGTC | CCAAGATCCAACTACGAGCTT |
| TLR 3 | GTATTGCCTGGTTTGTTAATTGG | AAGAGTTCAAAGGGGGCACT |
| IL-1 | CGCCAATGACTCAGAGGAAG | AGGGCGTCATTCAGGATCAA |
| G-CSF | AGCTTCCTGCTCAAGTGC | TTCTTCCATCTGCTGCCAGATGGT |
| IL-12 | TGGAGTGCCAGGAGGACAGT | TCTTGGGTGGGTCAGGTTTG |
| IFN-alpha | GACTCCATCTTGGCTGTGA | TGATTTCTGCTCTGACAACCT |

### Example 2

### Human Lung Organoid Models of SARS-CoV-2 Infection

New human lung organoid models were developed to model infection, including SARS-CoV-2. The in vitro modeling of interactions between infectious agents and their target tissues has typically utilized human cancer cell lines. However, recent 3D organoid methods have greatly facilitated the robust primary culture of diverse wild-type human tissues, affording a much more physiologic in vitro infectious disease platform. The topologic self-organization of organoids maintains numerous biological properties of primary tissues, such as self-renewal, multilineage differentiation, signaling nodes, histology, and pathology. Once established, organoids can be cultured long term, expanded, cryopreserved, and genetically manipulated similar to traditional 2D cell lines. As such, organoids combine the tractability of in vitro systems with 3D architecture and differentiation of in vivo organisms, as highly relevant to infectious disease modeling.

Pathogenicity of respiratory diseases are attributable to local inflammation and, in extreme cases, systemic cytokine storm. The establishment of a holistic 3D in vitro pathogen infection model, encompassing both epithelial and immune components is largely unexplored, yet critical for devising new countermeasures against microbial diseases. Here, we described 3D human lung ALI organoids as the first ex vivo culture system inclusive of epithelial and endogenous immune cells for studying enteric and respiratory infectious diseases. This incorporates BSL2/BSL3 infection protocols, single cell transcriptomic, and spatial single cell IHC technologies to provide an integrative system for modeling the immune consequences of epithelial SARS-CoV-2, infection that is extendable to any lung infectious pathogen.

The COVID-19 pandemic has arisen within the unforeseen and exponential onset of SARS-CoV-2 coronavirus human transmission. Since the beginning of the 21st century, three emerging human coronaviruses have evolved from bats including SARS-CoV in 2003, Middle East Respiratory Coronavirus (MERS-CoV) in 2012 and SARS-CoV-2 in 2019, the etiologic agent of COVID-19. SARS-CoV caused about 8,000 infections and 800 deaths, while the MERS-CoV outbreak is still ongoing and has resulted in about 2500 cases and 35% mortality. In contrast, SARS-CoV-2 has caused over a million cases worldwide resulting in 62,000 deaths in a rapidly expanding pandemic. In the US, over 500,000 deaths have occurred as of 2021. A clear understanding of the targets of viral infection in the lung and the consequences of viral infection in different lung compartments and cell communities is essential for devising new countermeasures for global health.

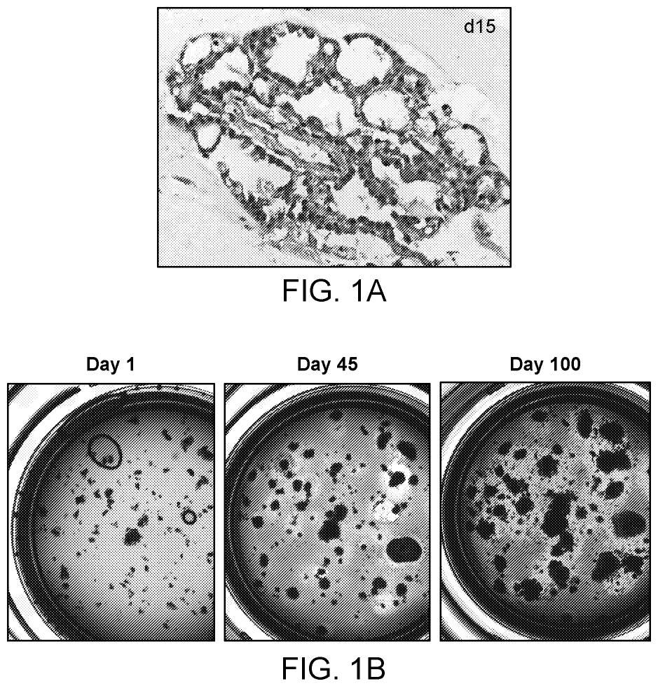

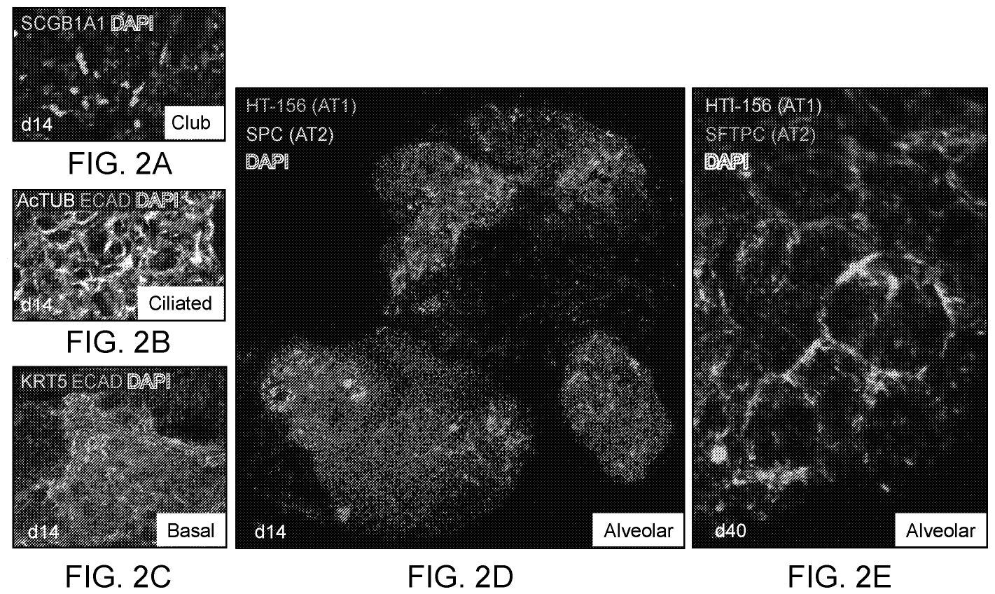

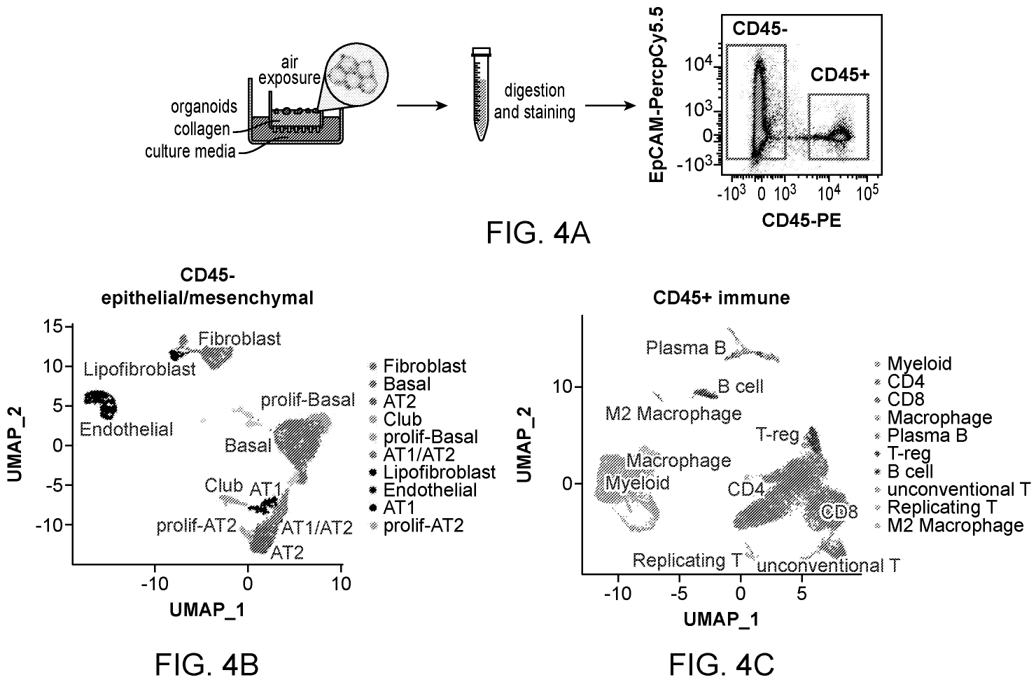

Air-liquid interface organoids preserving epithelial and immune cells en bloc. Conventional organoids are grown in Matrigel-like ECM and submerged beneath tissue culture media, but exclusively contain epithelial cells without immune components. Unfortunately, such exclusively epithelial organoids cannot model the excessive inflammation that is a hallmark of lethal SARS-CoV-2 infection. Here we have generated novel human lung 3D ALI organoids that strongly contrast with post-mitotic monolayer 2D airway cultures. The novel human lung 3D ALI organoids expand for >100 days (longest time attempted), can be generated from small endoscopic biopsies, and contain fibroblast, endothelial and neural stroma as well as diverse immune cells (T, B, macrophages) (FIG. 1-4). Our studies indicate that human lung ALI cultures can indeed be generated, histologically preserve alveolar air spaces, and retain endogenous immune cells such as T cells and plasma cells (FIG. 1-4). Here, these ALI lung organoids are used to explore immune consequences of epithelialSARS-CoV-2 infection.

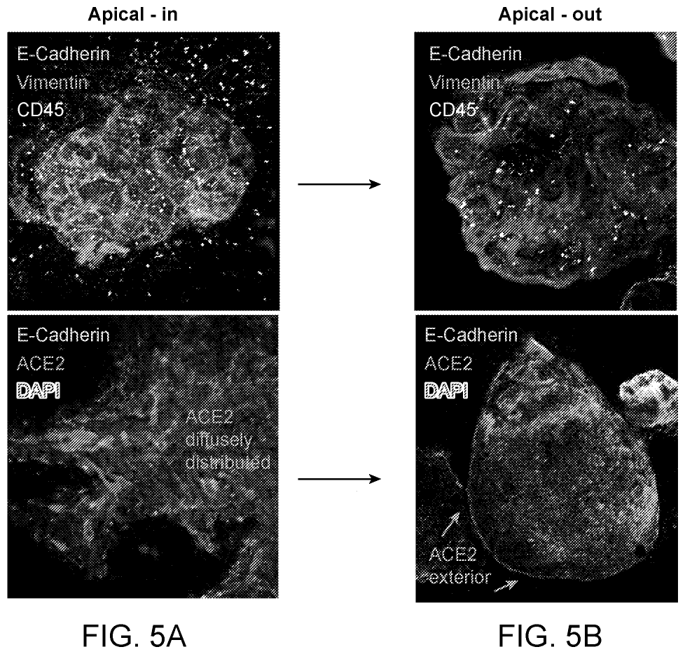

Regulated apical-basal polarity in 3D lung ALI organoids. Organoids grow as epithelial spheroids encased in an extracellular matrix (ECM) scaffold. In traditional organoid culture technology, the apical side of the epithelium faces the center of the spheroid. That is, the surface that represents the lumen of the gut or airway is facing inward or has an “apical-in” topology. The natural mode of interaction between a virus and the epithelium is with the apical side of the epithelium facing the lumen of the gut or airway. Such constraints also exist for 3D lung ALI organoids in which the apical surface is are typically oriented towards the interior. Thus it is desirable to modify the 3D lung ALI organoids by turning them inside out such that the apical surface now faces the exterior tissue culture media and infectious agents such as SARS-CoV-2 can be easily added for infection of the apical aspect. We trigger a morphogenetic eversion event. by polarity of the epithelium is changed from “apical-in” to “apical-out”. We have tested and refined this method to create apical-out 3D ALI organoids from human lung where the ACE2 apical receptor for SARS-CoV-2 is expressed on the exterior of the organoid (FIG. 5).

BSL-3 RNA-seq. Single cell RNA-seq of organoids in a BSL3 setting is problematic because of the need for extensive cell disaggregation and processing in a stringent biosafety environment. We performed scRNA-seq within a Stanford BSL3 facility, allowing scRNA-seq of ALI organoids containing both epithelial and immune components.

Preparation of human 3D ALI lung organoids. 3D lung ALI organoids are routinely prepared from normal human lung tissue specimens. The peripheral 1 cm of the lung, containing alveoli and distal bronchioles, is minced and enzymatically processed and grown as submerged Matrigel domes. Human distal lung is washed with PBS, minced finely on ice, and resuspended in Cultrex Rat Collagen I ((R&D). 1 mL of tissue-collagen suspension is layered on top of pre-solidified 1 mL collagen gel within a 30 mm, 0.4 micron inner transwell. After fully solidifying, the 6 collagen transwells are placed in a standard 6-well tissue culture plate. 1 mL of lung ALI culture media (see below) is added into the tissue culture plate, below the bottom surface of the collagen-containing transwell. Media is changed 2×/week as described in Example 1.

Apical-basal polarity reversal. In one variation, the 3D ALI human lung organoids, which normally grow in a “basal-out” configuration, will undergo polarity reversal to an “apical-out” state. We can induce lung organoids to reverse polarity such that the apical surface faces the medium which would greatly facilitate apical pathogen entry. To evert, collagen was removed using Collagenase Type 4, resuspended in the lung ALI media and plated in a low-attachment 6-well plate (Corning) to restructure for 2-5 days as described in Example 1.

These apical-out organoids differentiate into multiple lung resident cells and can be infected in suspension, distributed into different wells to simultaneously expose the same culture to different variables, fixed for confocal whole mount 3D imaging or histology, or dispersed for scRNA-seq and other molecular analyses.

Prior methods by others have resorted to mechanical shearing to access the apical surface, and have neither used holistic immune-epithelial organoids nor used a suspension culture eversion method as we describe in the present application.

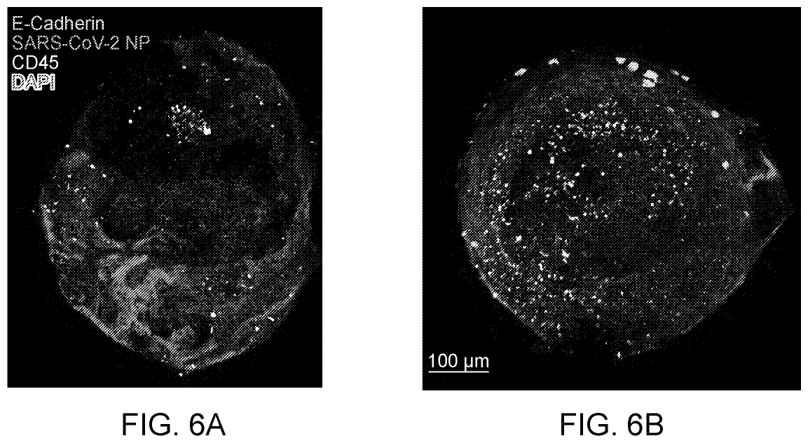

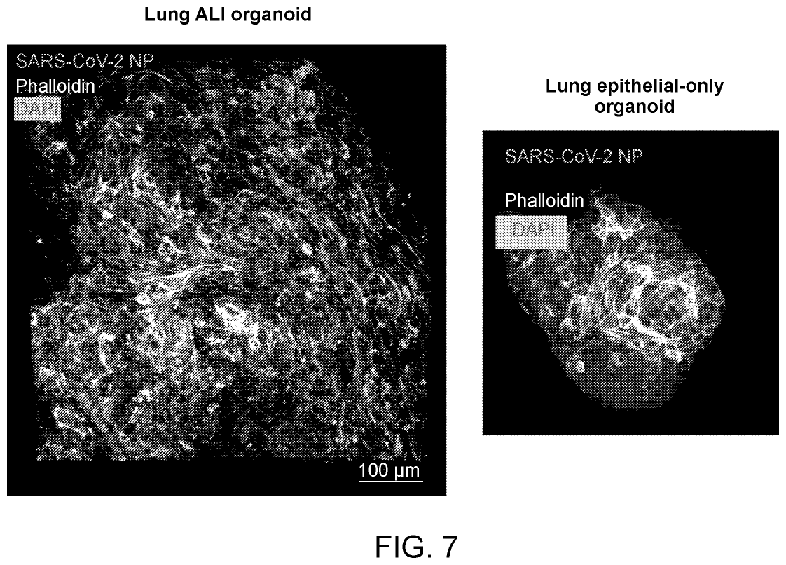

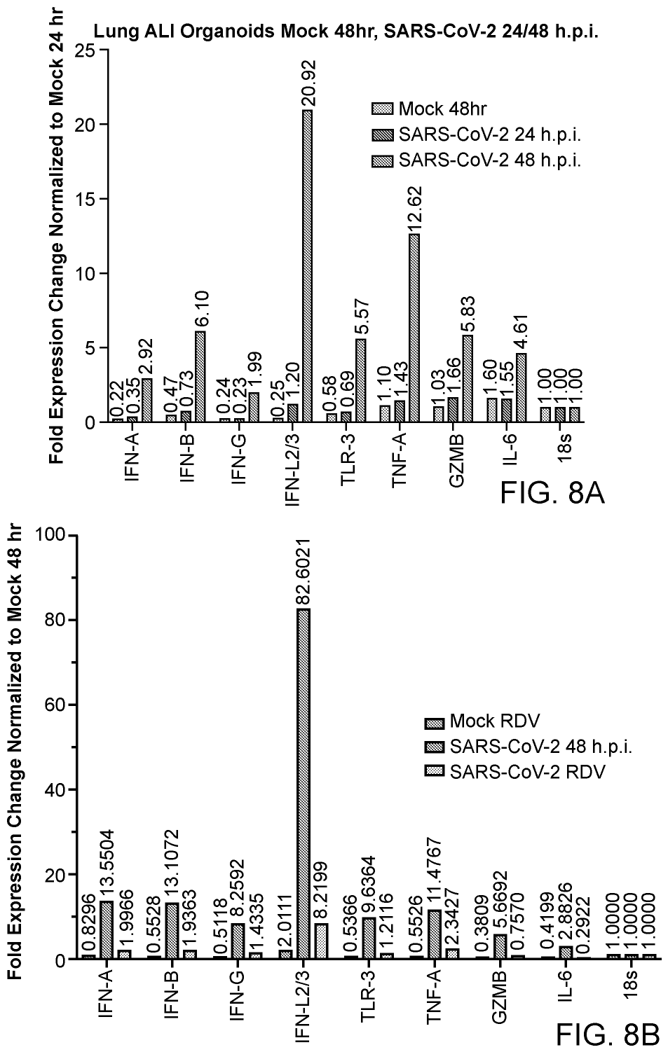

SARS-CoV-2 infection of lung ALI organoids in suspension. Human lung ALI organoids were grown in collagen for 5-10 days, put into suspension for 2-5 days, counted, then infected with SARS-CoV-2 prior to day 14 in culture. Organoids were resuspended in virus media or an equal volume of mock media, at a MOI of 1 relative to total organoid cells in the sample, and then incubated at 37° C. under 5% CO2 for 2 hours. Organoids were then plated in suspension in lung ALI organoid media At the indicated timepoints, organoids were washed with lung organoid media and PBS and either resuspended in TRIzol LS (Thermo Fisher), freshly-made 4% PFA in PBS. All SARS-CoV-2 work was performed in a class II biosafety cabinet under BSL3 conditions at Stanford University. For remdesivir experiments (RDV), organoids were infected with SARS-CoV-2 as described above, and spiked with 10 uM RDV at 24 hours post-infection (FIG. 6-8).

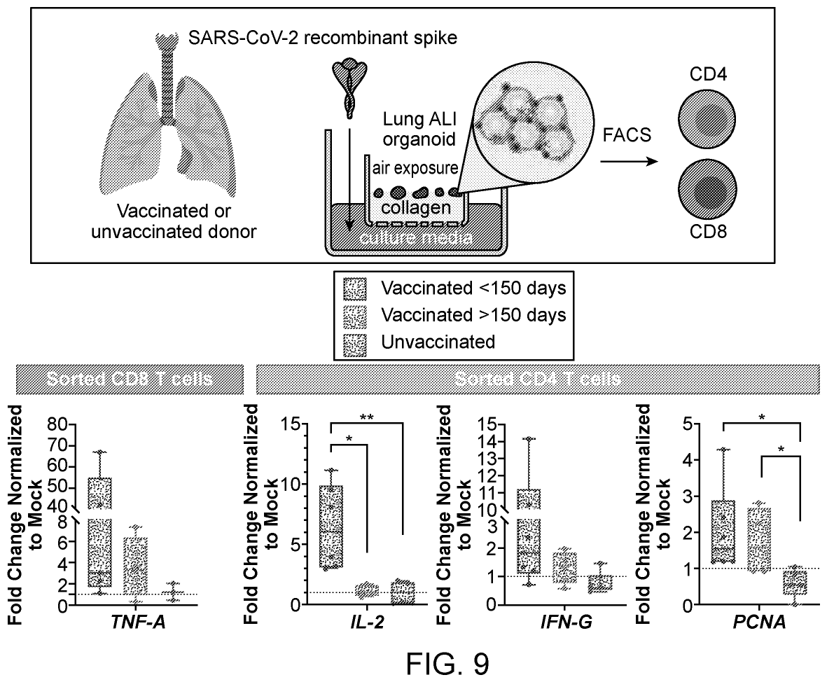

SARS-CoV-2 recombinant spike protein treatment of ALI organoids. Organoids were grown in collagen for 7-10 days. SARS-CoV-2 S1 subunit recombinant protein was added to cell culture media at a concentration of 60 ng/mL (reconstituted in PBS) for 4 days. Equivalent volume of PBS was added to cell culture media for the mock condition. After 4 days of stimulation, organoids were digested and processed for flow cytometry as described above. Cells were sorted into Extraction Buffer (RNA PicoPure kit) and RNA was isolated as per manufacture's instructions. RNA was processed for qRT-PCR determination of gene expression as above (FIG. 9).

GFP fluorescence analysis of live organoids. Cultures of organoids are infected for 1 hr and followed for 72 hrs postinfection in BSL3. Fluorescent images for SARS-CoV-2 infection are captured by microscopy and quantified using ImageJ²³. Typically, a collection of random images is taken at 0, 12, 24, 48 and 72 hrs post-infection.

Viral genome copy number determination. Quantitative RT-PCR approaches are used to measure the accumulation of viral genomes or mRNA after infection. Briefly, a subset of cultures is harvested at 2, 24, 48 and 72 hrs post-infection, RNA extracted and subjected to qRT-PCR.

Histologic analysis include H&E and 3D confocal imaging with staining for differentiation and polarity markers to determine preferred sites of viral entry, replication, and spread. This includes KRT5 (basal), SFTPC, HTII-280 (alveolar), AcTub (ciliated), SCGB1A1 (Club), ACE2 expression and topology, and SARS-CoV-2 double labeling with anti-GFP, anti-SARS-CoV-2 spike protein and anti-dsRNA antibodies (FIG. 6, 7).

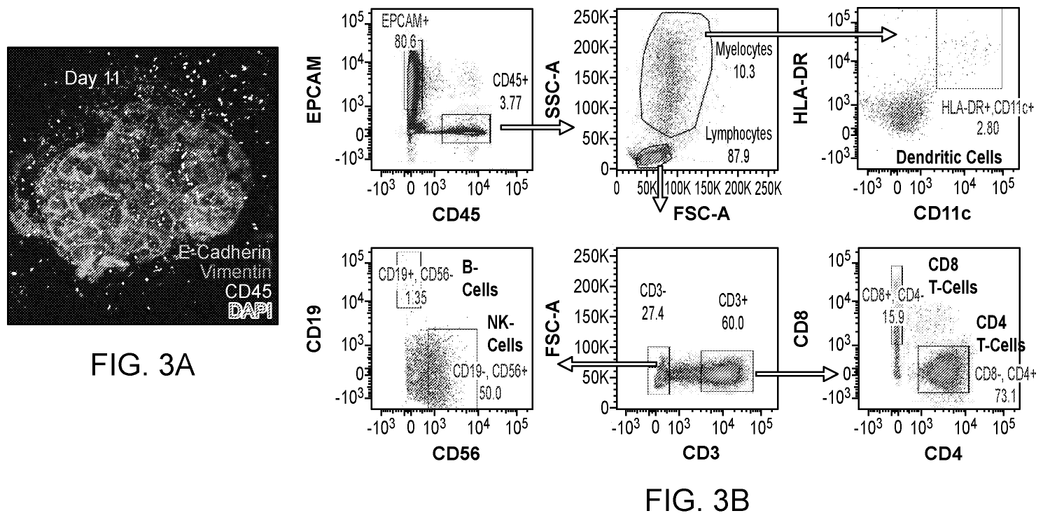

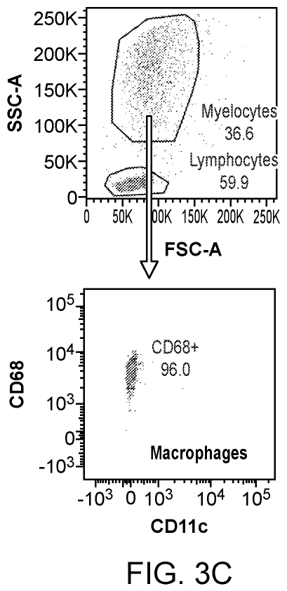

The pathogenicity of SARS-CoV-2 and the COVID-19 infection syndrome is highly attributable to local inflammation and systemic cytokine storm. The exploration of how primary infection of lung and/or intestinal epithelial cells results in this highly deleterious secondary immune activation would strongly benefit from a human in vitro system containing both epithelial and immune components. Our applications describes just such a human lung epithelial-immune system that robustly maintain both epithelial and immune populations en bloc as a cohesive unit without reconstitution. These ALI organoids preserve a diversity of immune cell types including T, B and myeloid cells (FIG. 3-5). This ALI model allows de novo initiation of SARS-CoV-2 infection in an in vitro human system allowing measurement of cross-talk between epithelial and immune components.

Single cell RNA-seq and TCR-seq of immune and epithelial compartments. Mock infected ALI organoids and three time points (i.e. early, intermediate and late, up to 96 h) undergo MACS-purification (Miltenyi Biotec) of EPCAM⁺ epithelial and CD45⁺ immune compartments followed by single cell 3′ RNA-seq for epithelium- and 5′ RNA/TCR-/BCR(Ig)-seq for the immune-compartment with the 10× Genomics Chromium whole gene expression or Immune Profiling system, and NextSeq/HiSeq sequencing. The resultant single cell 3′ or 5′ transcriptome and BCR/TCRαβ CDR3 sequences is analyzed by Cell Ranger and Seurat, followed by tSNE/UMAP visualizations to determine pathogen-specific expression profiles of (1) epithelium and (2) associated immune cell components. Validated bioinformatic workflows for single cell overlay of virus transcripts onto intestinal epithelium are used. The pseudotime algorithm defines trajectories of SARS-CoV-2-induced responses in epithelial and immune compartments.

Integrative scRNA-seq network modeling of lung epithelial and immune responses. 1. KEGG pathway mapper analyzes gene expression profiles from the intestinal epithelium vs immune subsets to map genes onto cytokine-receptor pathways. 2. NicheNet models intercellular communication by predicting ligand-target links between interacting cell types. This integrated network time course of human ALI organoid infection allows analysis of nature and timing of epithelial-immune crosstalk.

Imaging analysis for ALI organoid infection along with immune cells. In parallel, a time course spatial imaging analysis by CODEX is performed, simultaneously detecting 50+ markers in FFPE sections at single cell resolution. Characterizing pathogen-induced spatial changes in situ provides single cell analysis orthogonal to scRNA-seq for understanding the temporal immune response to infection.

SARS-CoV-2-GFP infection of distal lung ALI organoids enables performing longitudinal scRNA-seq analysis of propagation of SARS-CoV-2-GFP responses from primary epithelial infection to successive waves of cell type-specific immune responses using 5′ RNA/TCR-/BCR(Ig)-seq workflows templated for rotavirus. These lung ALI studies are of paramount importance for modeling SARS-CoV-2-induced inflammation in distal lung, a major cause of morbidity via respiratory failure, as well as SARS-CoV-2 cytokine storm. Further, lung ALI cultures are used to screen SARS-CoV-2 immunomodulatory therapeutics.

The scope of the SARS-CoV-2/COVID-19 pandemic, combined with the current lack of treatment options, has raised an urgent need for cell culture methods to screen potential therapeutics. Current screening methods can utilize SARS-CoV-2 infection of transformed cell lines (c.f. Vero, African green monkey kidney) that are not relevant to lung biology. Established post-mitotic 2D ALI transwell cultures of proximal airway epithelium are (1) difficult to scale and (2) do not recapitulate the distal airways (alveoli and bronchioles). In contrast, our organoid methods provide unique in vitro capabilities for SARS-CoV-2 therapeutics screening with the advantage of having both epithelial and immune compartments.

Further, the development of 3D lung ALI cultures provides a unique capability to assay therapeutics that block immune responses initiated by SARS-CoV-2 infection of lung epithelium, such as remdesivir or other further agents.

We can screen multiplexed 3D ALI human lung organoids. These are plated in a variety of formats ranging from 96 to 1536 well formats and encompass mixed, alveolar or basal/bronchiolar cultures. Multiplexed cultures are ported to BSL3 SARS-CoV-2-GFP and SARS-CoV-2-nLuc infection with optimized protocols and GFP/nLuc plate reader quantitation (Tecan). Ultimately, candidate therapeutics are overlaid onto multiplexed SARS-CoV-2-GFP/nLuc infection, including currently available (e.g. remdesavir, darunavir, nelfinavir) and novel therapeutics, e.g. anti-SARS-CoV-2 mAb, over 5-8 point concentration curves with S/B measurement of assay robustness.

Endpoints for screening 3D lung ALI cultures. The 3D lung ALI cultures allows novel endpoint screening for (1) primary lung epithelial infection and (2) secondary immune responses. Here, 3D lung ALI cultures undergo BSL3 infection with SARS-CoV-2 or other pathogens, followed by inhibitor treatment. This includes antivirals and mAbs but also antagonists of immune activation (c.f. anti-IL6R, Kevzara, Actemra) to model blunting of the inflammatory response (Nanostring, Luminex), but also to measure inhibition of lung epithelial damage (Annexin V) secondary to limiting cytokine storm that is particularly lethal to COVID-19 patients. Definition of such immune endpoints and cross-talk is uniquely enabled by our 3D ALI organoids with their seminal attribute of preserving both lung epithelial and diverse infiltrating immune cells en bloc without reconstitution.

Novel holistic methods for ALI modeling of any respiratory pathogenic infection incorporating epithelial and immune populations en bloc are developed. This allows infection-induced signaling cascades that occur between epithelium and immune cells, within immune cells, and their reciprocal feedback to epithelium. Infection-stimulated adaptive immune responses can be assessed from tissue-resident memory populations. Furthermore, we can identify pathogen-specific cell types susceptible to infection. Pathogen infection time can be iteratively modified and peripheral blood immune cells can be added. Candidate therapeutics and vaccines can be evaluated.

### REFERENCES

1. Huang, J. Y., et al. Chemodetection and Destruction of Host Urea Allows Helicobacter pylori to Locate the Epithelium. Cell Host Microbe 18, 147-156 (2015).
2. Ding, S., et al. Retinoic Acid and Lymphotoxin Signaling Promote Differentiation of Human Intestinal M Cells. Gastroenterology (2020).
3. Ding, S., et al. STAG2 deficiency induces interferon responses via cGAS-STING pathway and restricts virus infection. Nat Commun 9, 1485 (2018). PMC5902600
4. Co, J. Y., et al. Controlling Epithelial Polarity: A Human Enteroid Model for Host-Pathogen Interactions. Cell Rep 26, 2509-2520 e2504 (2019). PMC6391775
5. Neal, J. T., et al. Organoid Modeling of the Tumor Immune Microenvironment. Cell 175, 1972-1988 e1916 (2018).
6. Mou, H., et al. Dual SMAD Signaling Inhibition Enables Long-Term Expansion of Diverse Epithelial Basal Cells. Cell Stem Cell 19, 217-231 (2016). PMC4975684
7. Kretzschmar, K. & Clevers, H. Organoids: Modeling Development and the Stem Cell Niche in a Dish. Dev Cell 38, 590-600 (2016).
8. Hegab, A. E., et al. Isolation and in vitro characterization of basal and submucosal gland duct stem/progenitor cells from human proximal airways. Stem Cells Transl Med 1, 719-724 (2012). PMC3659656
9. Kumar, P. A., et al. Distal airway stem cells yield alveoli in vitro and during lung regeneration following H1N1 influenza infection. Cell 147, 525-538 (2011). PMC4040224
10. Zhou, J., et al. Differentiated human airway organoids to assess infectivity of emerging influenza virus. Proc Natl Acad Sci USA 115, 6822-6827 (2018). PMC6042130
11. Tata, P. R. & Rajagopal, J. Plasticity in the lung: making and breaking cell identity. Development 144, 755-766 (2017). PMC5374348
12. Sachs, N., et al. Long-term expanding human airway organoids for disease modeling. EMBO J (2019).
13. Barkauskas, C. E., et al. Type 2 alveolar cells are stem cells in adult lung. J Clin Invest 123, 3025-3036 (2013). PMC3696553
14. Barkauskas, C. E., et al. Lung organoids: current uses and future promise. Development 144, 986-997 (2017). 5358104
15. Zacharias, W. J., et al. Regeneration of the lung alveolus by an evolutionarily conserved epithelial progenitor. Nature 555, 251-255 (2018). PMC6020060
16. Dye, B. R., et al. In vitro generation of human pluripotent stem cell derived lung organoids. Elife 4(2015). PMC4370217
17. Chen, Y. W., et al. A three-dimensional model of human lung development and disease from pluripotent stem cells. Nat Cell Biol 19, 542-549 (2017).
18. Yamamoto, Y., et al. Long-term expansion of alveolar stem cells derived from human iPS cells in organoids. Nat Methods (2017).
19. Jacob, A., et al. Differentiation of Human Pluripotent Stem Cells into Functional Lung Alveolar Epithelial Cells. Cell Stem Cell 21, 472-488 e410 (2017).
20. Li, X., et al. Oncogenic transformation of diverse gastrointestinal tissues in primary organoid culture. Nat Med 20, 769-777 (2014). PMC4087144
21. Ootani, A., et al. Sustained in vitro intestinal epithelial culture within a Wnt-dependent stem cell niche. Nat Med 15, 701-706 (2009).
22. Scobey, T., et al. Reverse genetics with a full-length infectious cDNA of the Middle East respiratory syndrome coronavirus. Proc Natl Acad Sci USA 110, 16157-16162 (2013). PMC3791741
23. Schneider, C. A., Rasband, W. S. & Eliceiri, K. W. NIH Image to ImageJ: 25 years of image analysis. Nat Methods 9, 671-675 (2012). PMC5554542
24. Yan, K. S., et al. Intestinal Enteroendocrine Lineage Cells Possess Homeostatic and Injury-Inducible Stem Cell Activity. Cell Stem Cell 21, 78-90 e76 (2017).
25. Yan, K. S., et al. Non-equivalence of Wnt and R-spondin ligands during Lgr5+ intestinal stem-cell self-renewal. Nature 545, 238-242 (2017).
26. Sheahan, T. P., et al. Broad-spectrum antiviral GS-5734 inhibits both epidemic and zoonotic coronaviruses. Sci Transl Med 9(2017). PMC5567817
27. Sheahan, T. P., et al. Comparative therapeutic efficacy of remdesivir and combination lopinavir, ritonavir, and interferon beta against MERS-CoV. Nat Commun 11, 222 (2020). PMC6954302

### Example 3

Respiratory infectious disease research is limited by the lack of long-term human in vitro models of host-pathogen interactions and resultant inflammatory responses. This need has been exacerbated by the morbidity and mortality of the current SARS-CoV-2 pandemic. In particular, a significant gap exists regarding the activity of tissue-resident immune cells and their interaction with lung tissue in SARS-CoV-2 infection. Here, we describe the development of a novel human lung organoid system grown in a 3D air-liquid interface (ALI), which enables a holistic propagation of primary human lung epithelium replete with native stroma and tissue-resident immune cells without requiring artificial reconstitution. These lung ALI organoids contain endothelial, basal, club, alveolar type 1 and 2 (AT1, AT2) cells which remain proliferative for months in culture. Notably, the organoids also contain a diverse array of immune subsets, including helper and cytotoxic T cells, B cells, and macrophages. SARS-CoV-2 ALI organoid infection induced immune cell clustering around infected cells, and broad innate responses. Crucially, ALI lung organoids mounted adaptive CD4 and CD8 T cell responses to SARS-CoV-2 recombinant spike protein which absolutely correlated with patient donor immunization status and waned with time from vaccination. Overall, we establish a robust human lung organoid system preserving a full diversity of tissue-resident immune populations, allowing holistic analysis of SARS-CoV-2-induced pulmonary inflammation and representing an enabling method for respiratory infectious disease research.

### Results

Human lung ALI organoids grow robustly in culture and preserve distal lung histology. The cellular landscape of the human lung is rich and diverse, composed of different epithelial cell types, mesenchymal cells such as fibroblasts and endothelial cells, and a dynamic tissue-resident immune landscape. Our previously reported epithelial-only spheroids were created by first subjecting lung tissue to enzymatic dissociation and plating cells as single-cell suspension in Matrigel submerged beneath tissue culture media. These exclusively-epithelial organoids cannot model the excessive inflammation that is a hallmark of lethal SARS-CoV-2 infection. In contrast to the epithelial-only Matrigel organoids, the 3-D lung ALI organoids consist of tissue that is mechanically minced and kept as intact larger tissue fragments. The fragments are suspended in collagen within a trans-well with access to culture media on one side and air on the other. Culture media consisted of Advanced DMEM/F-12 supplemented with EGF, Noggin, fetal calf serum, Y-27632 (ROCK inhibitor), and CHIR-99021 (GSK-3 inhibitor). The increased oxygenation and lack of enzymatic dissociation allows for more cell types to be maintained and for tissue structure to remain intact. The organoids described in this work were generated from normal distal lung tissue of over 80 patients undergoing surgical lobectomies with a culture success rate of over 80%. The lung ALI organoids expanded in culture for up to 180 days (longest time attempted). H&E staining of a day 10 organoid showed cuboidal epithelium lining alveoli-like spaces.

Lung ALI organoids maintain diverse epithelial and mesenchymal makeup of distal lung. We first set out to fully characterize the cellular makeup of these novel organoids. Immunofluorescence imaging showed E-Cadherin+ epithelial cells closely associated with Vimentin+ mesenchymal cells and interspersed with CD45+ immune cells. Additionally, CD31 staining revealed endothelial cells forming network-like structures within the organoids (FIG. 1e). SFTPC+AT2 cells grew in distinct clusters within the organoids and remained proliferative with K167+ staining for over 60 day). Similar to the AT2 cells, KRT5+ basal cells also grew in distinct clusters, forming structures that resemble bronchioles extending into alveoli. Although spatially separated, AT2 cells and basal cells were seen within the same large organoid, contrasting with epithelial-only spheroids in which basal and alveolar organoids were only seen in mutually exclusive organoids. Additionally, ALI organoids contained rare SCGB1A1+ club cells and acetylated-tubulin+ ciliated cells. Notably, the ciliated cells only appeared after about three weeks in culture. This was similar to the epithelial-only spheroids in which ciliated cells appeared later in culture than other differentiated cells such as club cells. Lastly, the lung ALI organoids also contained alveoli-like cystic structures lined with HT1-56+AT1 cells.

After determining spatial relationships of cell types using confocal microscopy, we then used 10× droplet-based single-cell RNA sequencing to confirm cell populations. Organoids from 3 patients were grown for 12 days, dissociated, stained, and FACS-sorted for CD45− live, single cells in order to get an exclusively epithelial and mesenchymal dataset. After sequencing, multi-patient data were integrated for phenotyping. The proportion of epithelial cells compared to fibroblast and endothelial cells varied in the three patients with epithelial cells comprising the majority in all patients. The epithelial cells mostly comprised of KRT5+ basal cells with a small cluster of SCGB1A1+ club cells. AT2 cells were phenotyped by the co-expression of pneumocyte marker NKX2-1 and AT2 markers SFTPC and LAMP3. AT1 cells were phenotyped by the co-expression of NKX2-1 and AT1 marker HOPX1 and the absence of SFTPC/LAMP3. In all 3 patients, we observed a population of NKX2-1+/HOPX−/SFTPC− pneumocytes labeled AT1/AT2. These cells were not found in the patient-matched tissue sample, leading us to suspect their presence may be a result of the culture system. Notably, ciliated cells are absent from scRNA-sequencing data from organoids as cells were captured from day 12 in culture and ciliated cells appear after 3 weeks in culture.

Human lung ALI organoids retain diverse immune cell populations and TCR diversity. We next sought to characterize the immune cell content of the lung ALI organoids. Immunofluorescence imaging of CD3 and CD68 showed T-cells and macrophages embedded throughout the epithelium. In order to determine both relative proportions of immune cell populations and their longevity in culture, organoids were dissociated at various timepoints, stained with immune cell markers, and analyzed by flow cytometry. Although organoids from different patients had variable starting amounts of immune cells, relative proportions of each immune cell type were similar among patients. Earlier timepoints consisted mostly of lymphocytes, with CD4 T cells being most abundant followed by CD8 T cells and relatively rare B cells. As seen in our prior tumor ALI organoid work, without exogenous cytokine support lymphocyte populations decreased substantially over time, with virtually none remaining at day 33 and beyond. SSC-hi, CD68+, CD11c− macrophages began as a minority of total CD45+ cells but persisted over time and eventually comprised the majority of CD45+ cells. We next performed single-cell RNA sequencing of live, single, CD45+ FACS-sorted cells from three separate patients. Single-cell RNA sequencing of CD45+ cells from organoids at day 12 confirmed the CD4, CD8, B cell, and macrophage populations found by flow cytometry. Additionally, there was a small population of CD3E+, CD8−, NKG7+NK-T cells. There was also a small population of CD3E, CD4, FOXP3 positive Tregs and SDC1+ plasma cells. Notably, sequencing of CD45+ cells from a paired tissue-organoid sample revealed preservation of all major immune cell types with the exception of granulocytes. We observed a gene marker+ population of granulocytes, likely neutrophils and mast cells that are not retained in organoid culture. After confirming the presence of different immune cell types, we next sought to compare the TCR repertoire of T-cells from organoid culture and patient-matched tissue to determine whether organoids maintained immune diversity and T-cell receptor (TCR) repertoire of the tissue of origin. 5′ VDJ TCR sequencing of a patient-matched tissue and day 12 organoid pair revealed highly conserved TCR repertoires.

Removing ECM from lung ALI organoids allows for epithelial and stromal reorientation and apical ACE2 access. To facilitate viral access to ACE2-expressing epithelial cells (which are typically buried in layers of collagen and fibroblasts), we adapted our previously published method of removing ECM from the lung spheroids to have the epithelium “invert” out. In collagen, the epithelial cells form central primary structures surrounded by mesenchymal cells that migrate through the collagen. When we grew ALI organoids in collagen for ˜7 days and removed the ECM to culture them in suspension, the entire organoid was able to reorient the epithelium to the apical surface while mesenchymal cells and immune cells remained embedded in the epithelium. We performed single-cell RNA sequencing on CD45− and CD45+ cells in suspension to confirm all major cell types were still represented with verification by confocal microscopy. We then integrated the patient-matched collagen and suspension CD45−/+data sets to confirm no major transcriptomic changes occurred during the suspension culture process. Crucially, suspension culture allowed ACE2-expressing epithelial cells to be exposed on the apical surface of the organoids without being obstructed by layers of stroma and collagen matrix.

Human lung ALI organoids can be infected with SARS-CoV-2 for extended times. Using our suspension culture, we next set out to infect the organoids with SARS-CoV-2. The suspension culture allowed viral access to epithelial cells while retaining immune cells (FIG. 3d left and right). A huge advantage of this system is that cells can persist for a longer time than other ex vivo culture systems. SARS-CoV-2 infection has been shown to induce multinucleate syncytia. Synctitia were observed in the infected organoids at later timepoints. At later timepoints post-infection there is immune cell clustering around area of infection. Lots of infection at later timepoints, overlaps well with cleaved caspase-3, showing that infected cells are under apoptotic stress. This was quantified.

Lung ALI organoids initiate dynamic responses to SARS-CoV-2. Target cells types include mostly AT2 cells, club cells, rare ciliated cells. There was no difference at early (3 day) or late (10 day) timepoints post-infection. Bulk RNA-sequencing of whole organoids at different timepoints shows dynamic changes in gene processes including chemotaxis genes for T-cells, macrophages, neutrophils. Expression of immunoglobulin genes, including mucosal IgA was seen. Other genes expressed include Interferon-stimulated genes; Coagulation factors, which may be relevant to coagulopathies associated with mortality. Luminex of media from time points. Secreted CD40 ligand and IL-2 from interaction of CD4 T-cells with effector cells such as macrophages and B-cells. IL-10 increased in patient serum with severity of infection. TNF-A, IFN-G. Innate cytokine such as IL-1 and IL-6, also associated with morbidity and mortality in patient serum. Chemotaxis ligand (RANTES) to attract T-cells, associated with mortality in patient serums. TGF-A and PDGF-AA associated with fibroblast activity.

We then wanted to test whether patients developed antigen-specific memory T cells in the lung in response to vaccination. We cultured organoids from vaccinated and unvaccinated patients. We also collected blood and tested serum for presence of Spike IgG with ELISA to confirm immunization status. We added the SARS-CoV-2 spike S1 subunit recombinant protein to cell culture media for 4 days, then dissociated the organoids and sorted for CD4 and CD8 T-cells for qPCR. We were surprised to find differences in markers of antigen-specific T-cell activation, such as IL-2, IFN-G, and TNF-A in vaccinated patients when stratified by length of time since last vaccine dose (before or after 150 days). The T-cells from vaccinated patients who received dosages over 150 days ago behaved more similarly to those of unvaccinated patients. Waning immunity post-vaccination may be an explanation for why booster shots are important.

Although SARS-CoV-2-induced systemic inflammation has been well characterized by studies of patient-derived peripheral immune cells and sera, the progression of lung tissue-level events remains elusive. Current in vitro models consist of immortalized cell lines that do not retain multiple cell types or tissue explants with limited survival over several days. Our prior lung spheroid system allowed us to model multi-cell-type infection of the human distal lung but was limited to the lung epithelium. In this current work, we develop a novel human lung organoid system using a 3-D air-liquid-interface to retain not only multiple types of epithelial cells (basal, club, AT1, AT2) but also mesenchymal and tissue-resident immune cells. This new organoid system retains helper, cytotoxic, and even regulatory T-cells along with B/plasma cells and macrophages.

Notably, infection of lung ALI organoids with SARS-CoV-2 induced pronounced time-dependent cleaved-caspase-3 expression in infected cells and clustering of immune cells around the infected area. As the presence of ciliated cells were time-restricted in these organoids, we observed only rare ciliated cell infection. Instead, infection was limited to primarily AT2 cells and club cells. Extended time courses of infection (3-14 days) revealed induction of pathways related to immune cell chemotaxis, immunoglobulin production, interferon-stimulated genes, and even angiogenesis and coagulation. Lastly, we observed time-since-vaccination dependent differences in T-cell response to recombinant spike protein. This implies that tissue-level T-cell immunity to SARS-CoV-2 spike protein declines after vaccination, a trend that reflects increases in SARS-CoV-2 infection rates in vaccinated patients over time and decreases in infection rates among those recently boosted. This may provide rationale for the necessity of further boosters if immunity amongst the vaccinated population continues to decline.

### Methods

Human distal lung culture: Human distal lung was washed with PBS, minced finely on ice, and resuspended in Cultrex Rat Collagen I ((R&D). 1 mL of tissue-collagen suspension was layered on top of pre-solidified 1 mL collagen gel within a 30 mm, 0.4 mm inner transwell. After fully solidifying, the 6 collagen transwells were placed in a standard 6-well tissue culture plate. 1 mL of lung ALI culture media (see below) was added into the tissue culture plate, below the bottom surface of the collagen-containing transwell. Media was changed 2×/week.

Lung ALI culture media: Advanced DMEM/F12 (Invitrogen) supplemented with 10 mM nicotinamide, n-acetyl cysteine, 1×B27 supplement minus vitamin A, recombinant human NOGGIN (100 ng/mL, R&D Systems), recombinant human EGF (50 ng/mL, R&D Systems), and TGF-beta inhibitor A83-01 (100 nM, Tocris). This mixture was then supplemented with 10% fetal bovine serum, Y-27632 (Peprotech), CHIR 99021 (R&D).

Histologic analysis of ALI organoids: Collagen from transwell containing ALI organoids were fixed in 10% formalin for 1 hr at room temp, cut into thin slices, and placed into a histology cassette with 70% ethanol. The collagen was then paraffin embedded and sectioned (4-5 mm). Sections were deparaffinized and stained with H&E for histological analysis.

Serial brightfield imaging: Tissue culture plates containing the ALI organoids in transwells were imaged serially with a Keyence microscope. Images were stitched using BZ-Wide viewer software.

Whole-mount staining and confocal microscopy of ALI organoids: collagen containing ALI organoids was cut away from the transwell and fixed in 4% PFA for 1 hour at room temp. PFA was neutralized with 1×PBS-glycine for 30 min at room temp, then blocked and permeabilized with 10% donkey serum in 1×IF buffer for 2 hours at room temp. Organoids were then stained with primary antibodies at room temp for 3 days overnight, followed by 3×30 min washes with 1×IF wash buffer. Secondary staining was carried out with 1:1000 fluorescent donkey secondaries (Jackson Immunoresearch) and DAPI for 4 hours at room temp, then washed 3×30 min. Organoids were then mounted on slides with mounting buffer (Prolong Gold Antifade mounting media, Thermo). Images were acquired using Zeiss LSM-900 confocal microscope, and viewed in 3-D using Imaris software.

List of Primary Antibodies:

| | | | |
|---|---|---|---|
| Antigen | Supplier | Product Number | Dilution |

| | | |
|---|---|---|
| Ki-67 | | 1:50 |
| E-cadherin | Abcam | 1:300 |
| E-cadherin | BD | 1:200 |
| Krt5-Alexa488 | Abcam | 1:200 |
| SFTPC | | 1:200 |
| SCGB1A1 | | 1:300 |
| Ac-Tub | | 1:200 |
| HT1-56 | | 1:200 |
| CD45 | | 1:300 |
| CD3 | | 1:100 |
| Vimentin | | 1:300 |
| ACE2 | | 1:100 |
| SARS-CoV-2 | | 1:200 |
| Nucleocapsid Protein | | |
| Cleaved Caspase-3 | | 1:400 |

Staining buffer recipes:

### 10×PBS:Glycine (500 mL):

### 38.00 g NaCl

9.38 g Na₂HPO₄
2.07 g NaH₂PO₄

### 37.50 g Glycine

Fill to 500 mL with 1×PBS
pH 7.4 and filter sterilize
10×IF-permeabilization buffer (500 mL)

### 38.00 g NaCl

9.38 g Na₂HPO₄
2.07 g NaH₂PO₄

### 2.5 g NaN₃

### 5.0 g BSA (Fraction V)

### 10.00 mL Triton X-100

2.5 mL tween-20
Fill to 500 mL with 1×PBS
pH 7.4 and filter sterilize

FACS analysis of immune surface markers from lung ALI organoids: Collagen containing lung ALI organoids was digested with Collagenase Type 4 (Worthington), for 30 min with shaking at 37 degrees C., then centrifuged and washed with media containing FBS to quench collagenase. Organoids (now dissociated from collagen) were digested to single-cell with Liberase TL (Sigma) and DNAse (Worthington) for 30 min with shaking at 37 degrees C., and washed and quenched with FACS buffer (5 mM EDTA+5% FBS). Cells were stained for viability with Zombie Aqua (Biolegend) 1:500 in FACS buffer for 20 min on ice, protected from light. After washing with FACS buffer, cells were stained for surface markers with antibodies listed below, all at 1:100. Compensation was performed using OneComp eBeads™ Compensation Beads (Thermo) and primary antibodies 1:400. Sorting and analysis was performed on a BD FACSAria II SORP, with further data analysis in FlowJo.

Eversion of human lung ALI organoids: Lung ALI organoids were grown as previously described in collagen for 5-10 days. To evert, collagen was removed using Collagenase Type 4 (Worthington) for 30 min with shaking at 37 degrees C. Collagenase was washed and quenched with FBS containing media 3×, for 10 min each at room temp. Organoids were collected by centrifuging at 100G for 3 min at room temp. They were then resuspended in the lung ALI media (previously described) and plated in 1.5-2 mL each in a low-attachment 6-well plate (Corning), and allowed to restructure for 2-5 days.

Single-cell RNA sequencing of human lung ALI organoids: Organoids were harvested either from collagen or suspension, digested into single-cell as previously described, and sorted on a BD FACSAria II SORP for singlet discrimination, followed by live/dead gating, followed by CD45−/+ gating. Cellular suspensions were loaded on a Chromium Single Cell Controller instrument (10× Genomics, Pleasanton, CA, USA) to generate single-cell GEMs. Libraries for sequencing was prepped as per manufacturer's instructions using a 5′ library prep kit (10× Genomics).

SARS-CoV-2 infection of lung ALI organoids in suspension: VeroE6 cells were obtained from ATCC and maintained in supplemented DMEM with 10% FBS. SARS-CoV-2 (USA-WA1/2020) was passaged in VeroE6 cells in DMEM with 2% FBS. Titers were determined by plaque assay on VeroE6 cells using Avicel (FMC Biopolymer) and crystal violet (Sigma), viral genome sequence was verified, and all infections were performed with passage 3 virus. Human lung ALI organoids were grown in collagen for 5-10 days, put into suspension for 2-5 days, counted, then infected with SARS-CoV-2 prior to day 14 in culture. Organoids were resuspended in virus media or an equal volume of mock media, at a MOI of 1 relative to total organoid cells in the sample, and then incubated at 37° C. under 5% CO₂ for 2 hours. Organoids were then plated in suspension in lung ALI organoid media At the indicated timepoints, organoids were washed with lung organoid media and PBS and either resuspended in TRIzol LS (Thermo Fisher), freshly-made 4% PFA in PBS. All SARS-CoV-2 work was performed in a class II biosafety cabinet under BSL3 conditions at Stanford University. For remdesivir experiments (RDV), organoids were infected with SARS-CoV-2 as described above, and spiked with 10 uM RDV at 24 hours post-infection.

qPCR analysis of SARS-CoV-2 RNA. RNA from SARS-CoV-2-infected organoids was extracted by adding 750 μl TRIzol (Thermo Fisher Scientific), and purified using an RNA Clean & Concentrator-25 kit (Zymo Research) as per manufacturer instructions. All RNA samples were treated with DNase (Turbo DNA-free kit, Thermo Fisher Scientific). The Brilliant II SYBR Green QRT-PCR 1-Step Master Mix (VWR) was used to convert RNA to cDNA and amplify specific RNA regions on the CFX96 Touch real-time PCR detection system (Bio-Rad). RT reaction was performed for 30 min at 50° C., 10 min at 95° C., followed by two-step qPCR with 95° C. for 10 seconds and 55° C. for 30 seconds, for a total of 40 cycles. Primer sequences are as follows:

| Gene | Forward primer | Reverse primer |
|---|---|---|
| TNF-alpha | TCTTCTCGAACCCCGAGTGA | CCTCTGATGGCACCACCAG |
| IL-6 | GTAGCCGCCCCACACAGACAGCC | GCCATCTTTGGAAGGTTC |
| M-CSF | GGAGACCTCGTGCCAAATTA | TATCTCTGAAGCGCATGGTG |
| 18S | GGCCCTGTAATTGGAATGAGTC | CCAAGATCCAACTACGAGCTT |
| TLR 3 | GTATTGCCTGGTTTGTTAATTGG | AAGAGTTCAAAGGGGGCACT |
| IL-1 | CGCCAATGACTCAGAGGAAG | AGGGCGTCATTCAGGATCAA |
| G-CSF | AGCTTCCTGCTCAAGTGC | TTCTTCCATCTGCTGCCAGATGGT |
| IL-12 | TGGAGTGCCAGGAGGACAGT | TCTTGGGTGGGTCAGGTTTG |
| IFN-alpha | GACTCCATCTTGGCTGTGA | TGATTTCTGCTCTGACAACCT |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for culture of organoid cultures with distal lung alveoli and bronchioles en bloc with infiltrating endogenous immune cells and mesenchymal cells as a cohesive unit without artificial reconstitution, comprising:
initiating a culture with a fragment of distal lung tissue;
culturing the cells in a 3D air-liquid interface (ALI) with culture medium comprising extracellular matrix and an effective concentration of factors, for a period of time sufficient to form organoids comprising alveolar and terminal bronchiolar cells and endogenous immune and stromal cells.

2. The method of claim 1, wherein the organoids comprise lung epithelial, mesenchymal and immune cells.

3. The method of claim 2, wherein the epithelial cells comprise E-cadherin⁺ epithelial cells, SFTPC⁺ AT2 cells, KRT5⁺ basal cells, SCGB1A1⁺ club cells, tubulin⁺ cilia cells, and HT1-56⁺ AT1 cells.

4. The method of claim 2, wherein the immune cells comprise CD4⁺ T cells, CD8⁺ T cells, B cells, and macrophages.

5. The method of claim 2, where the mesenchymal cells are vimentin⁺ mesenchymal cells.

6. The method of claim 1, wherein the distal lung tissue is human.

7. The method of claim 1, wherein the organoid is infected with a respiratory pathogen.

8. The method of claim 7, wherein the respiratory pathogen in a virus.

9. The method of claim 8, wherein the virus is influenza virus; adenovirus; human bocavirus; human coronavirus including SARS-CoV1 and SARS-CoV2; human metapneumovirus; human parainfluenza virus; human respiratory syncytial virus; or human rhinovirus.

10. The method of claim 7, wherein the respiratory pathogen is a bacteria.

11. The method of claim 10, wherein the bacteria is Mycobacterium tuberculosis, Streptococcus pneumoniae, Mycoplasma pneumoniae, Haemophilus influenzae, Chlamydophila pneumoniae; Chlamydia psittaci; Coxiella burnetiid; Legionella pneumophila, Staphylococcus aureus; or Klebsiella pneumoniae.

12. The method of claim 1, wherein the organoids are everted by the process of:
removing organoids from extracellular matrix culture;
placing the organoids in suspension culture, thereby leading to relocation of differentiated cells from the lumen of the organoid, to the organoid exterior.

13. The method of claim 1, wherein the organoids are viable in culture for a period of at least 28 days.

14. The method of claim 1, wherein the factors in the culture medium comprise epidermal growth factor (EGF) and a BMP antagonist.

15. The method of claim 1, wherein the BMP antagonist is NOGGIN protein.

16. The method of claim 1, wherein the medium further comprises an inhibitor of TGF-b.

17. The method of claim 1, further comprising contacting the organoid with a candidate agent for an effect on lung tissue’ and
determining the effect of the agent on one or more cells present in the organoid.

18. The method of claim 17, wherein the candidate agent is an anti-viral agent.

19. The method of claim 17, wherein the agent is a vaccine, optionally in combination with an adjuvant.

20. (canceled)

21. The method of claim 1, comprising the step of analyzing the organoid by one or more of single cell RNA sequencing, microscopy, including fluorescence microscopy and staining, confocal imaging; quantitative RT-PCR; spatial imaging analysis of sections at single cell resolution, measuring viability.

22. (canceled)


SEQUENCE LISTING (18 sequences)

SEQ ID NO: 1 - DNA, 20 bp, Artificial sequence
  synthetic sequence
tcttctcgaaccccgagtga

SEQ ID NO: 2 - DNA, 23 bp, Artificial sequence
  synthetic sequence
gtagccgccccacacagacagcc

SEQ ID NO: 3 - DNA, 20 bp, Artificial sequence
  synthetic sequence
ggagacctcgtgccaaatta

SEQ ID NO: 4 - DNA, 22 bp, Artificial sequence
  synthetic sequence
ggccctgtaattggaatgagtc

SEQ ID NO: 5 - DNA, 23 bp, Artificial sequence
  synthetic sequence
gtattgcctggtttgttaattgg

SEQ ID NO: 6 - DNA, 20 bp, Artificial sequence
  synthetic sequence
cgccaatgactcagaggaag

SEQ ID NO: 7 - DNA, 18 bp, Artificial sequence
  synthetic sequence
agcttcctgctcaagtgc

SEQ ID NO: 8 - DNA, 20 bp, Artificial sequence
  synthetic sequence
tggagtgccaggaggacagt

SEQ ID NO: 9 - DNA, 19 bp, Artificial sequence
  synthetic sequence
gactccatcttggctgtga

SEQ ID NO: 10 - DNA, 19 bp, Artificial sequence
  synthetic sequence
cctctgatggcaccaccag

SEQ ID NO: 11 - DNA, 18 bp, Artificial sequence
  synthetic sequence
gccatctttggaaggttc

SEQ ID NO: 12 - DNA, 20 bp, Artificial sequence
  synthetic sequence
tatctctgaagcgcatggtg

SEQ ID NO: 13 - DNA, 21 bp, Artificial sequence
  synthetic sequence
ccaagatccaactacgagctt

SEQ ID NO: 14 - DNA, 20 bp, Artificial sequence
  synthetic sequence
aagagttcaaagggggcact

SEQ ID NO: 15 - DNA, 20 bp, Artificial sequence
  synthetic sequence
agggcgtcattcaggatcaa

SEQ ID NO: 16 - DNA, 24 bp, Artificial sequence
  synthetic sequence
ttcttccatctgctgccagatggt

SEQ ID NO: 17 - DNA, 20 bp, Artificial sequence
  synthetic sequence
tcttgggtgggtcaggtttg

SEQ ID NO: 18 - DNA, 21 bp, Artificial sequence
  synthetic sequence
tgatttctgctctgacaacct